SEQUENCE LISTING (29 sequences)

SEQ ID NO: 1 - protein, 615 aa, Melanocarpus albomyces
METVNTTARLAALRSLMKEKGVDVYIVPSEDSHSSEYIAACDARRAFISGFTGSAGTAVVTHDKAALATDGRYFNQAGKQLDSNWTLLKTGMQDVPTWQEWTAEESAGGKTVGVDPTLIASSVAEKLDESVKKSGGAGLKAVDENLVDLVWGADRPARSNNPVVLLPEKYTGKDTAAKLADLRKELDKKKASAFVLSMLDEIAWLFNLRGSDITYNPVFFSYAIVTRDSATLYVDASKLDAEARSYLDQNKVAIKPYGDLYRDAQALASTAEADKAGERPTKYLMSNKGSWALKLALGGDKFVEEIRSPVADAKAVKNDVELDGMRKCHIRDGAALIEFFAWLEDQLVNKKAVIDEVAAADKLEELRRKQKDFVGPSFDTISSTGPNAAIIHYKPERGNCAVIDPNAIYLCDSGAQYLDGTTDVTRTLHFGTPTAEEKKAYTLVLKGNISLDTAVFPKGTTGLAIDCLARQHLWKAGLDYRHGTGHGVGSYLNVHEGPIGIGTRKQYADVALAAGNVLSIEPGYYEDGVYGIRIENLAIVREVKTEYTFDDKPFLGFEHVTMVPYCRRLIDESLLTADEKQWLNKANQEIRANMEGYFKDDELTRSWLERETQPF

SEQ ID NO: 2 - protein, 612 aa, Malbrancheae cinamonea
METVDTSQRLADLRKLMKQYSVDVYIIPSEDSHQSEYIAPCDARRAFISGFTGSAGIAIVSMTKAALSTDGRYFNQASRQLDNNWTLLKRGIEGYPTWQEWTAEQSQGGKVVGVDPTLITTADSRQLSDQLKSSGGKLIGVSDNLVDLVWGKDRPARPNEKVRVHPIELAGKSAEEKIEDLRKELEKKKKAGIIISMLDEIAWLFNLRGNDIPYNPVFFSYALVTQSTAELYIDEDKLSPEVRAHLGDKITIKPYGAIFSVARALSQSSAGDSGDGSQKFLLSNKASWALNLALGGDVRVDEIRSPIADAKAIKNDAELKGMRACHIRDGAALTEYFAWLENELVNKGTVIDEVQASDKLEEIRSKHKNFVGLSFDTISSTGPNAAVIHYKAERGNCSIIDPKAIYLCDSGAQYLDGTTDTTRTLHFGEPTEMEKRAYTLVLKGMISIDTAVFPKGTTGYAIDAFARQHLWREGLDYLHGTGHGVGSYLNVHEGPMGLGTRPQYAEIPLAAGQVISDEPGYYEDGNFGIRIENVVIVKEVETPYKFGSRPYLGFEHVTMTPLCRKLIEPSLLTAQEKQWVNDYHAEVWEKTSGYFENDELTRNWLKRETAPI

SEQ ID NO: 3 - protein, 653 aa, Trichoderma citrinoviride
MYRPLVAAAPSLAFRFPRKLPGQFISRLATVAMGRANTTQKLAKLRALMKEHNVQVYVVPSEDSHSSEYIAACDARREFISGFTGSAGCAVITETAAALATDGRYFNQATQQLDENWTLLKQGLQDVPTWQEWAAEQSAGGKKVAVDSTLITASIAKKLAEKIRKSGGSDLVPLDVNLVDAVWAEDRPARPQQRITVLSEKFAGKSVQAKLSDVFSELEKKRSPGLFISMLDEVAWLFNLRGNDIPYNPVFFSYAVITPKGAALYVDESKLDEECREHLNKCNVAIKPYDSFFRDAELLHQQFVASTQSAEGAASAAGSFLMSNRGSWALKRALGGEGAVEEVRSPIGDAKAIKNETEMEGMRACHIRDGAALIEYFAWLEDQLINKKTVLDEVQAADKLEELRSKHEHFVGLSFPTISSTGANAAVIHYGPERGNCATIDPKAIYLCDSGAQYLDGTTDTTRTLHFGEPSEAEREAYTLVLKGNIALDVAVFPKGTTGFALDSLARQHLWQNGLDYRHGTGHGVGSFLNVHEGPIGIGTRIQYTEVPLAPGNVISNEPGYYEDGRFGIRIENIIMVKEVKTKYAFGDKPFLGFEHVTMVPYCRNLINESMLSEAEKAWLNASNAEILEKTKGFFEGDALTMAWLTRETRPIE

SEQ ID NO: 4 - protein, 652 aa, Fusarium verticillioides
MLFQTATSLLRSAPRRLAAASRLSSRRWASSENMTKLDTTSRLNRLRGLMKERNVQIYIVPSEDSHSSEYIADCDARRAYISGFTGSAGCAVVTLESAALATDGRYFNQATSQLDSNWTLLKQGLQDVPTWQDWSAEQSSGGKNVGVDPTLISGSTAKNLAEKIRKNGGAELLPVDGNLVDLVWGDERPSRPSEQVIIQPDELAGESVLNKLTKVRQELEKKHSPGFLVSMLDEIAWLFNLRGNDIPYNPVFFAYATVTPDAAKLYIDEAKLDDKCRSHLTSNKVDIKPYETIFDDAQALHAAHAAKSKSGDKVPTGNFLISNKGSWALKRALGGDSSVDEIRSLIGDAKAIKTEAELKGMRDCHVRDGAALIQYFAWLEDQLVNKKATLDEVQAADKLEELRKVKKDFVGLSFPTISSTGANAAIIHYGPERGNCATIDPEAIYLCDSGAQYRDGTTDTTRTLHFGKPTEAEREAYTLVLKGHISLDQAIFPKGTTGFALDSLARQHLWKNGLDYRHGTGHGVGSFLNVHEGPIGIGTRVQYAEVALAPGNVLSNEPGYYEDGKYGIRIENMVLVKEVKTKHSFGDKPFLGFEYVTLVPYCRNLIDTTLLTSEEKEWLNTYNAKVLEKTQEYFEGDDVTLAWLKRETQHVE

SEQ ID NO: 5 - protein, 647 aa, Stagonospora sp.
MLARCLRRTHVAVRHSSPSPRTFHASPALRAIDMAKVDTTERLAQLRKLMKERNVDVYMVPSEDSHQSEYIAPCDARRAYISGFTGSAGYAVVTHEKAALSTDGRYFNQAEKQLDSNWELLKQGIQDVPTIQEWTADQVEGGKVVGVDPSVVTAADARKLADKIKKKGGEYKAIDENLVDLVWGAERPARPSEKVLVQPLEYSGKSFDDKIDDLRKELEKKKSLGFVVSMLDETAWLLNLRGNDIPYNPVFFSYAVVTPTAVTLYVDESKLPDEVKSHLSDKVTVRPYDAIFDDVAVLSKEAFAASGEADSQKKFLTSNRASWALNKALGGEDKVEETRSPIGDAKAVKNETELEGMRQCHIRDGAAISEYFAWLEDQLLNKKATLDEVDGADKLEAIRKKHDKFMGLSFDTISSTGANAAVIHYKPEKGACSIIDPAAIYLCDSGAQYHDGTTDTTRTLHFTKPTDMEKKAYTLVLKGNIALERVKFPKGTTGFALDAIARQFLWAEGLDYRHGTGHGVGSFLNVHEGPIGIGTRVQYSEVSLAVGNVISDEPGYYEDGKFGIRIENMVMVKEVETNHKFGDKPYLGFEHVTLTPHCRNLVDMGLLTKDEKEFINAYHQEVFDKTSKFFENDSVTLEWLKRETAPY

SEQ ID NO: 6 - DNA, 138 bp, Synthesized
atgcagaccttcggtgcttttctcgtttccttcctcgccgccaggtaagtagacactcactggaattcgttcctttcccgatcatcatgaaagcaagtagactgactgaaccaaacaactagcggcctggccgcggcc

SEQ ID NO: 7 - protein, 633 aa, Fusarium verticillioides
ASRLSSRRWASSENMTKLDTTSRLNRLRGLMKERNVQIYIVPSEDSHSSEYIADCDARRAYISGFTGSAGCAVVTLESAALATDGRYFNQATSQLDSNWTLLKQGLQDVPTWQDWSAEQSSGGKNVGVDPTLISGSTAKNLAEKIRKNGGAELLPVDGNLVDLVWGDERPSRPSEQVIIQPDELAGESVLNKLTKVRQELEKKHSPGFLVSMLDEIAWLFNLRGNDIPYNPVFFAYATVTPDAAKLYIDEAKLDDKCRSHLTSNKVDIKPYETIFDDAQALHAAHAAKSKSGDKVPTGNFLISNKGSWALKRALGGDSSVDEIRSLIGDAKAIKTEAELKGMRDCHVRDGAALIQYFAWLEDQLVNKKATLDEVQAADKLEELRKVKKDFVGLSFPTISSTGANAAIIHYGPERGNCATIDPEAIYLCDSGAQYRDGTTDTTRTLHFGKPTEAEREAYTLVLKGHISLDQAIFPKGTTGFALDSLARQHLWKNGLDYRHGTGHGVGSFLNVHEGPIGIGTRVQYAEVALAPGNVLSNEPGYYEDGKYGIRIENMVLVKEVKTKHSFGDKPFLGFEYVTLVPYCRNLIDTTLLTSEEKEWLNTYNAKVLEKTQEYFEGDDVTLAWLKRETQHVE

SEQ ID NO: 8 - protein, 622 aa, Stagonospora sp.
SPALRAIDMAKVDTTERLAQLRKLMKERNVDVYMVPSEDSHQSEYIAPCDARRAYISGFTGSAGYAVVTHEKAALSTDGRYFNQAEKQLDSNWELLKQGIQDVPTIQEWTADQVEGGKVVGVDPSVVTAADARKLADKIKKKGGEYKAIDENLVDLVWGAERPARPSEKVLVQPLEYSGKSFDDKIDDLRKELEKKKSLGFVVSMLDETAWLLNLRGNDIPYNPVFFSYAVVTPTAVTLYVDESKLPDEVKSHLSDKVTVRPYDAIFDDVAVLSKEAFAASGEADSQKKFLTSNRASWALNKALGGEDKVEETRSPIGDAKAVKNETELEGMRQCHIRDGAAISEYFAWLEDQLLNKKATLDEVDGADKLEAIRKKHDKFMGLSFDTISSTGANAAVIHYKPEKGACSIIDPAAIYLCDSGAQYHDGTTDTTRTLHFTKPTDMEKKAYTLVLKGNIALERVKFPKGTTGFALDAIARQFLWAEGLDYRHGTGHGVGSFLNVHEGPIGIGTRVQYSEVSLAVGNVISDEPGYYEDGKFGIRIENMVMVKEVETNHKFGDKPYLGFEHVTLTPHCRNLVDMGLLTKDEKEFINAYHQEVFDKTSKFFENDSVTLEWLKRETAPY

SEQ ID NO: 9 - protein, 5 aa, Synthesized
QPQQP

SEQ ID NO: 10 - protein, 486 aa, Aspergillus clavatus
NAPGGPGGHGRKLPVNPKTFPNEIRLKDLLHGSQKLEDFAYAYPERNRVFGGQAHLDTVNYLYRELKKTGYYDVYKQPQVHQWTRADQSLTLGGDSIQASTMTYSPSVNVTAPLSLVSKLGCAEGDYSADVKGKIALVSRGECSFAQKSVLSAKAGAVATIVYNNVDGSLAGTLGGATSELGPYSPIIGITLAAGQDLVARLQAAPTEVSLWIDSKVENRTTYNVIAQTKGGDPNNVVALGGHTDSVENGPGINDDGSGVISNLVVAKALTRYSVKNAVRFCFWTAEEFGLLGSNYYVDNLSPAELAKIRLYLNFDMIASPNYALMIYDGDGSAFNLTGPPGSAQIESLFENYYKSIKQGFVPTAFDGRSDYEGFILKGIPAGGVFTGAESLKTEEQARLFGGQAGVALDANYHAKGDNMTNLNHKAFLINSRATAFAVATYANNLSSIPPRNATVVKRESMKWTKREEPHTHGADTGCFASRVKE

SEQ ID NO: 11 - protein, 483 aa, Neosartorya fischeri
NGPGWDWKPPVHPKVLPQMIHLWDLMHGAQKLEDFAYAYPERNRVFGGPAHEDTVNYLYRELKKTGYYDVYKQPQVHQWTRADQALTVDGKSYVATTMTYSPSVNVTAPLAVVNNLGCVESDYPADLKGKIALVSRGECPFATKSVLSAKAGAAAALVYNNIEGSMAGTLGGPTSELGPYAPIAGISLADGQALIQMIQAGTVTANLWIDSKVENRTTYNVIAQTKGGDPNNVVALGGHTDSVEAGPGINDDGSGIISNLVVAKALTRFSVKNAVRFCFWTAEEFGLLGSSYYVNSLNATEKAKIRLYLNFDMIASPNYALMIYDGDGSAFNLTGPAGSAQIERLFEDYYKSIRKPFVPTEFNGRSDYEAFILNGIPAGGIFTGAEAIKTEEQAKLFGGQAGVALDANYHAKGDNMTNLNREAFLINSKATAFAVATYANSLDSIPSRNMSTVVKRSQLEQAKKSTPHTHTGGTGCYKDRVEQ

SEQ ID NO: 12 - protein, 492 aa, Myceliophthora thermophila
GGHGGSSGLGCDSQRPLVSSEKLQSLIKKEDLLAGSQELQDIATAHGGHRAFGSSGHNATVDFLYYTLKALDYYNVTKQPFKEIFSSGTGSLTVDGEDIEAETLTYTPSGSATDKPVVVVANVGCDAADYPAEVAGNIALIKRGTCTFSQKSVNAKAAGAVAAIIYNNAEGKLSGTLGQPFLDYAPVLGITLEAGEALLAKLAGGPVTATLQIDALVEERVTYNVIAETKEGDHSNVLVLGGHTDSVPAGPGINDDGSGTIGMLTVAKALTKFRVKNAVRFAFWSAEEYGLLGSYAYIKSINSSAAELSKIRAYLNFDMIASPNYIYGIYDGDGNAFNLTGPAGSDVIERNFENFFKRKHTPSVPTEFSGRSDYAAFIENGIPSGGLFTGAEVLKTEREAELFGGRAGVAYDVNYHQAGDTVDNLALDAFLLNTKAIADSVATYALSFDGLPRVDGKKRRWDAHRARMLKRSAGSHGHAHLHSGPCGGGASI

SEQ ID NO: 13 - protein, 474 aa, Fusarium oxysporum
TKKPLVNELKLQKDINIKDLMAGAQKLQDIAEANGNTRVFGGAGHNATVDYLYKTLKATGYYNVKKQPFTELYSAGTASLKVDGDDITAAIMTYTPAGEATGPLVVAENLGCEASDFPAESEGKVVLVLRGECPFSQKSTNGKTAGAAAVIVYNNVPGELAGTLGEPFGEFAPIVGISQEDGQAILAKTKAGEVTVDLKVDATVENRVTFNVIAETKEGDHDNVLVVGGHSDSVAAGPGINDDGSGIIGILKVAQALTKYRVKNAVRFGFWSAEEFGLLGSYAYMKSINGSDAEVAKIRAYLNFDMIASPNYVYGIYDGDGSAFNLTGPAGSDAIEKDFERFFKTKRLGYVPSEFSGRSDYAAFIENGIPSGGLFTGAEQLKTEEEAKKFGGEAGVAYDINYHKIGDDINNLNKEAFLVNTQAIANSVARYAKTWKSLPKVTHNTRRWDAEVASVLKRSSGHSHAGGPCGSVSV

SEQ ID NO: 14 - protein, 488 aa, Fusarium oxysporum
LQIPLNLQVPKLSWNLFGDDLPLVDTKELQKSIKPENLEARAKDLYEIAKNGEEEYGHPTRVIGSEGHLGTLSYIHAELAKLGGYYSVSNQQFPAVSGNVFESRLVIGDSVPKQASPMGLTPPTKNKEPVHGTLVLVDNEGCDASDYPEAVKGNIALILRGTCPFGTKSGNAGKAGAVAAVVYNYEKDEVHGTLGTPSPDHVATFGLGGEEGKAVAKKLKDGEKVDAIAYIDAEVKTISTTNIIAQTRGGDPDNCVMLGGHSDSVAEGPGINDDGSGSISVLEVAVQLTKYRVNNCVRFAWWAAEEEGLLGSDHYVSVLPEDENRKIRLFMDYDMMASPNFAYQIYNATNAENPKGSEELRDLYVNWYEEQGLNYTFIPFDGRSDYDGFIRGGIPAGGIATGAEGVKTEDEVEMFGGEAGVWYDKNYHQIGDDLTNVNYTAWEVNTKLIAHSVATYAKSFKGFPEREIETSVQTYSDKTKYHGSKLFI

SEQ ID NO: 15 - protein, 494 aa, Chaetomium thermophilum var thermophilum DSM 1495
GGPHGFGLPKIDLRPMVSSNRLQSMITLKDLMDGAKKLQDIATKNGGNRAFGGAGHNATVDYLYKTLTSLGGYYTVKKQPFKEIFSSGSGSLIVDGQGIDAGIMTYTPGGSATANLVQVANLGCEDEDYPAEVAGNIALISRGSCTFSSKSLKAKAAGAVGAIVYNNVPGELSGTLGTPFLDYAPIVGISQEDGQVILEKLAAGPVTATLNIDAIVEERTTYNVIAETKEGDHNNVLIVGGHSDSVAAGPGINDDGSGTIGILTVAKALAKANVRIKNAVRFAFWSAEEFGLLGSYAYMKSLNESEAEVAKIRAYLNFDMIASPNYIYGIYDGDGNAFNLTGPAGSDIIEKDFEDFFKKKKTPSVPTEFSGRSDYAAFIENGIPSGGLFTGAEVLKTEEEAKLFGGKAGVAYDVNYHKAGDTVDNLAKDAFLLNTKAIANSVAKYAASWAGFPKPSAVRRRYDADMAQLLKRSGGVHGHGPHTHSGPCGGGDLL

SEQ ID NO: 16 - protein, 488 aa, Aspergillus terreus
EGLGNHGRKLDPNKFTKDIKLKDLLKGSQKLEDFAYAYPERNRVFGGKAHQDTVNWIYNELKKTGYYDVYKQPQVHLWSNAEQSLTVDGEAIDATTMTYSPSLKETTAEVVVVPGLGCTAADYPADVAGKIALIQRGSCTFGEKSVYAAAANAAAAIVYNNVDGSLSGTLGAATSELGPYAPIVGISLADGQNLVSLAQAGPLTVDLYINSQMENRTTHNVIAKSKGGDPNNVIVIGGHSDAVNQGPGVNDDGSGIISNLVIAKALTKYSLKNSVTWAFWTAEEFGLLGSEFYVNSLSAAEKDKIKLYLNFDMIASPNYALMIYDGDGSTFNMTGPAGSAEIEHLFEDYYKSRGLSYIPTAFDGRSDYEAFILNGIPAGGLFTGAEQIKTEEQVAMFGGQAGVAYDPNYHAAGDNMTNLSEEAFLINSKATAFAVATYANSLESIPPRNATMSIQTRSASRRAAAHRRAAKPHSHSGGTGCWHTRVEL

SEQ ID NO: 17 - protein, 486 aa, Aspergillus nidulans FGSC A4
GKHKPLVTPEALQDLITLDDLLAGSQQLQDFAYAYPERNRVFGGRAHDDTVNWLYRELKRTGYYHVYKQPQVHLYSNAEESLTVNGEAIEATTMTYSPSANASAELAVISGLGCSPADFASDVAGKVVLVQRGNCTFGEKSVYAAAADAAATIVYNNVEGSLSGTLGAAQSEQGPYSGIVGISLADGEALLALAEEGPVHVDLWIDSVMENRTTYNVIAQTKGGDPDNVVTLGGHSDSVEAGPGINDDGSGIISNLVIARALTKFSTKHAVRFFFWTAEEFGLLGSDYYVSSLSPAELAKIRLYLNFDMIASPNYGLLLYDGDGSAFNLTGPAGSDAIEKLFYDYFQSIGQATVETEFDGRSDYEAFILNGIPAGGVFTGAEEIKSEEEVALWGGEAGVAYDANYHQVGDTIDNLNTEAYLLNSKATAFAVATYANDLSTIPKREMTTAVKRANVNGHMHRRTMPKKRQTAHRHAAKGCFHSRVEQ

SEQ ID NO: 18 - protein, 274 aa, Bacillus licheniformis
AQTVPYGIPLIKADKVQAQGFKGANVKVAVLDTGIQASHPDLNVVGGASFVAGEAYNTDGNGHGTHVAGTVAALDNTTGVLGVAPSVSLYAVKVLNSSGSGSYSGIVSGIEWATTNGMDVINMSLGGASGSTAMKQAVDNAYARGVVVVAAAGNSGSSGNTNTIGYPAKYDSVIAVGAVDSNSNRASFSSVGAELEVMAPGAGVYSTYPTNTYATLNGTSMASPHVAGAAALILSKHPNLSASQVRNRLSSTATYLGSSFYYGKGLINVEAAAQ

SEQ ID NO: 19 - protein, 275 aa, Bacillus amyloliquefaciens
AQSVPYGVSQIKAPALHSQGYTGSNVKVAVIDSGIDSSHPDLKVAGGASMVPSETNPFQDNNSHGTHVAGTVAALNNSIGVLGVAPSASLYAVKVLGADGSGQYSWIINGIEWAIANNMDVINMSLGGPSGSAALKAAVDKAVASGVVVVAAAGNEGTSGSSSTVGYPGKYPSVIAVGAVDSSNQRASFSSVGPELDVMAPGVSIQSTLPGNKYGALNGTSMASPHVAGAAALILSKHPNWTNTQVRSSLENTTTKLGDSFYYGKGLINVQAAAQ

SEQ ID NO: 20 - protein, 269 aa, Bacillus lentus
AQSVPWGISRVQAPAAHNRGLTGSGVKVAVLDTGISTHPDLNIRGGASFVPGEPSTQDGNGHGTHVAGTIAALDNSIGVLGVAPSAELYAVKVLGASGSGAISSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 21 - protein, 316 aa, Geobacillus caldoproteolyticus
ITGTSTVGVGRGVLGDQKNINTTYSTYYYLQDNTRGNGIFTYDAKYRTTLPGSLWADADNQFFASYDAPAVDAHYYAGVTYDYYKNVHNRLSYDGNNAAIRSSVHYSQGYNNAFWNGSQMVYGDGDGQTFIPLSGGIDVVAHELTHAVTDYTAGLIYQNESGAINEAISDIFGTLVEFYANKNPDWEIGEDVYTPGISGDSLRSMSDPAKYGDPDHYSKRYTGTQDNGGVHINSGIINKAAYLISQGGTHYGVSVVGIGRDKLGKIFYRALTQYLTPTSNFSQLRAAAVQSATDLYGSTSQEVASVKQAFDAVGVK

SEQ ID NO: 22 - protein, 299 aa, Bacillus amyloliquefaciens
ATTGTGTTLKGKTVSLNISSESGKYVLRDLSKPTGTQIITYDLQNREYNLPGTLVSSTTNQFTTSSQRAAVDAHYNLGKVYDYFYQKFNRNSYDNKGGKIVSSVHYGSRYNNAAWIGDQMIYGDGDGSFFSPLSGSMDVTAHEMTHGVTQETANLNYENQPGALNESFSDVFGYFNDTEDWDIGEDITVSQPALRSLSNPTKYGQPDNFKNYKNLPNTDAGDYGGVHTNSGIPNKAAYNTITKIGVNKAEQIYYRALTVYLTPSSTFKDAKAALIQSARDLYGSQDAASVEAAWNAVGL

SEQ ID NO: 23 - protein, 387 aa, Trichoderma reesei (Hypocrea jecorina)
LPTEGQKTASVEVQYNKNYVPHGPTALFKAKRKYGAPISDNLKSLVAARQAKQALAKRQTGSAPNHPSDSADSEYITSVSIGTPAQVLPLDFDTGSSDLWVFSSETPKSSATGHAIYTPSKSSTSKKVSGASWSISYGDGSSSSGDVYTDKVTIGGFSVNTQGVESATRVSTEFVQDTVISGLVGLAFDSGNQVRPHPQKTWFSNAASSLAEPLFTADLRHGQNGSYNFGYIDTSVAKGPVAYTPVDNSQGFWEFTASGYSVGGGKLNRNSIDGIADTGTTLLLLDDNVVDAYYANVQSAQYDNQQEGVVFDCDEDLPSFSFGVGSSTITIPGDLLNLTPLEEGSSTCFGGLQSSSGIGINIFGDVALKAALVVFDLGNERLGWAQK

SEQ ID NO: 24 - protein, 212 aa, Ananas comosus
AVPQSIDWRDYGAVTSVKNQNPCGACWAFAAIATVESIYKIKKGILEPLSEQQVLDCAKGYGCKGGWEFRAFEFIISNKGVASGAIYPYKAAKGTCKTDGVPNSAYITGYARVPRNNESSMMYAVSKQPITVAVDANANFQYYKSGVFNGPCGTSLNHAVTAIGYGQDSIIYPKKWGAKWGEAGYIRMARDVSSSSGICGIAIDPLYPTLEE

SEQ ID NO: 25 - protein, 322 aa, Aspergillus niger
SAVTTPQNNDEEYLTPVTVGKSTLHLDFDTGSADLWVFSDELPSSEQTGHDLYTPSSSATKLSGYTWDISYGDGSSASGDVYRDTVTVGGVTTNKQAVEAASKISSEFVQDTANDGLLGLAFSSINTVQPKAQTTFFDTVKSQLDSPLFAVQLKHDAPGVYDFGYIDDSKYTGSITYTDADSSQGYWGFSTDGYSIGDGSSSSSGFSAIADTGTTLILLDDEIVSAYYEQVSGAQESEEAGGYVFSCSTNPPDFTVVIGDYKAVVPGKYINYAPISTGSSTCFGGIQSNSGLGLSILGDVFLKSQYVVFNSEGPKLGFAAQA

SEQ ID NO: 26 - protein, 223 aa, Sus scrofa
IVGGYTCAANSVPYQVSLNSGSHFCGGSLINSQWVVSAAHCYKSRIQVRLGEHNIDVLEGNEQFINAAKIITHPNFNGNTLDNDIMLIKLSSPATLNSRVATVSLPRSCAAAGTECLISGWGNTKSSGSSYPSLLQCLKAPVLSDSSCKSSYPGQITGNMICVGFLEGGKDSCQGDSGGPVVCNGQLQGIVSWGYGCAQKNKPGVYTKVCNYVNWIQQTIAAN

SEQ ID NO: 27 - protein, 230 aa, Bos taurus
IVNGEEAVPGSWPWQVSLQDKTGFHFCGGSLINENWVVTAAHCGVTTSDVVVAGEFDQGSSSEKIQKLKIAKVFKNSKYNSLTINNDITLLKLSTAASFSQTVSAVCLPSASDDFAAGTTCVTTGWGLTRYTNANTPDRLQQASLPLLSNTNCKKYWGTKIKDAMICAGASGVSSCMGDSGGPLVCKKNGAWTLVGIVSWGSSTCSTSTPGVYARVTALVNWVQQTLAAN

SEQ ID NO: 28 - protein, 481 aa, Aspergillus oryzae
GRALVSPDEFPEDIQLEDLLEGSQQLEDFAYAYPERNRVFGGKAHDDTVNYLYEELKKTGYYDVYKQPQVHLWSNADQTLKVGDEEIEAKTMTYSPSVEVTADVAVVKNLGCSEADYPSDVEGKVALIKRGECPFGDKSVLAAKAKAAASIVYNNVAGSMAGTLGAAQSDKGPYSAIVGISLEDGQKLIKLAEAGSVSVDLWVDSKQENRTTYNVVAQTKGGDPNNVVALGGHTDSVEAGPGINDDGSGIISNLVIAKALTQYSVKNAVRFLFWTAEEFGLLGSNYYVSHLNATELNKIRLYLNFDMIASPNYALMIYDGDGSAFNQSGPAGSAQIEKLFEDYYDSIDLPHIPTQFDGRSDYEAFILNGIPSGGLFTGAEGIMSEENASRWGGQAGVAYDANYHAAGDNMTNLNHEAFLINSKATAFAVATYANDLSSIPKRNTTSSLHRRARTMRPFGKRAPKTHAHVSGSGCWHSQVEA

SEQ ID NO: 29 - protein, 309 aa, Bacillus amyloiquefaciens
MVCRHYQELDAFVQEAKKKTGEGKVADYIPALAESGQDSLSVTIYHAENTCLTAGDADRTFTLQSISKVLSLALVLMDYGKEKVFSCVGQEPTGDPFNSMIKLETVNPGKPLNPMINAGALVVTSLIKGHSPKDRLNYLLGFIRRLANNQEITYCRHVAESEFSSSMINRAMCYYMKQYGIFKGDVEEVMDLYTKQCAIKMSSLDLAKIGCVFALDGKHPETGEQVIKKDVARICKTFMVTCGMYNASGEFAIKVGIPAKSGVSGGIMGISPYNFGIGIFGPALDEKGNSIAGVKLLEIMSEKYRLSIF

(19) United States
(12) Patent Application Publication
(10) Pub. No.: US 2022/0136027 A1
(43) Pub. Date: May 5, 2022

(54) PROTEIN HYDROLYSATES WITH INCREASED YIELD OF N-TERMINAL AMINO ACID

(71) Applicant: DUPONT NUTRITION BIOSCIENCES APS, COPENHAGEN K (DK)

(72) Inventors: Peter Edvard DEGN, Copenhagen K (DK); Xiaogang GU, Copenhagen K (DK); Karsten Matthias KRAGH, Copenhagen K (DK); Robin Anton SORG, Copenhagen K (DK); Steffen Yde BAK, Copenhagen K (DK); Svend HAANING, Copenhagen K (DK); Xinyue TANG, Copenhagen K (DK); Helong HAO, Copenhagen K (DK); Marc Anton Bernhard KOLKMAN, Copenhagen K (DK)

(21) Appl. No.: 17/433,767

(22) PCT Filed: Feb. 25, 2020
(86) PCT No.: PCT/US2020/019598

§ 371 Date: Aug. 25, 2021

(30) Foreign Application Priority Data
Feb. 25, 2019 (CN) PCT/CN2019/076018

(51) Int. Cl. C12P 21/06; A23J 3/34; A23J 3/18

(52) U.S. Cl. CPC C12P 21/06; A23J 3/18; A23J 3/346; C12Y 304/11009

(57) ABSTRACT

The present invention related to a method for preparing a protein hydrolysate from a proteinaceous material by contacting the material with a proteolytic enzyme mixture having a proline specific exopeptidase. In particular, the proline specific exopeptidase is an aminopeptidase specific for at the five amino acid N-terminal sequence X-Pro-Gln-Glv-Pro-, where X is any amino acid. The present invention also relates to use of the aminopeptidase with a second exopeptidase and an endopeptidase.

### TECHNICAL FIELD

The present invention relates to protein hydrolysates having an increased yield of the N-terminal amino acid where the penultimate N-terminal amino acid is proline. More particularly, the present invention relates to the use of amino peptidases with specificity for proline in the penultimate N terminal position for producing hydrolysates having an increased yield of free amino acids.

### BACKGROUND

Many food products such as soups, sauces and seasonings contain flavoring agents obtained by hydrolysis of proteinaceous materials. Conventionally, protein hydrolysates were generated by hydrolyzing proteinaceous materials such as defatted soy flour or wheat gluten with hydrochloric acid (HCl) at high temperature, typically under refluxing conditions. HCl generated protein hydrolysates are both flavorful and cheap. However, HCl treatment of proteins is also known to generate chlorohydrins such as monochlorodihydroxypropanols (MCDPs) and dichloropropanols (DCPs) which are perceived as potential health risks for consumers. See, e.g., J Velisek, J Davidek, et al., New Chlorine-Containing Organic Compounds in Protein Hydrolysates, J. Agric. Food Chem. 28, 1142-1144 (1980).

Possible health risks associated with chemical hydrolysis of proteins has led to the development of enzymes for use in producing tasty and low-cost protein hydrolysates. To ensure a high degree of hydrolysis, enzymatic procedures for making protein hydrolysates employ two non-specific proteases. First, a non-specific endoprotease is used to make internal cleavages in the protein or peptide. Next, the protein fragments generated by the endoprotease can be degraded into amino acids, dipeptides or tripeptides using exopeptidases. Non-specificity of the endoprotease is important to generate as many starting points as possible for the exoprotease. In this regard, amino-terminal peptidases cleave off amino acids, dipeptides or tripeptides from the amino terminal end of a protein or peptide. Carboxy-terminal peptidases cleave amino acids or dipeptides from the carboxy terminal end. It is understood in the art that non-specific exoproteases are also important so that as many amino acids as possible get removed from either the N or C terminus.

For protein hydrolysates intended for flavoring, the presence of glutamic acid (Glu) is crucial for flavor and palatability. In this regard, glutamine (Gln) is virtually tasteless whereas the corresponding Glu is tasty and provides a desirable taste. In conventional HCl proteolysis, deamidation, takes place without further steps. However, where enzymatic proteolysis is carried out, a glutaminase must be used which converts glutamine to glutamic acid.

There is a continuing need for methods and enzymes to produce protein hydrolysates having high levels of glutamic acid.

### SUMMARY OF THE INVENTION

In accordance with an aspect of the present invention, a method is presented for preparing a protein hydrolysate from a proteinaceous material in which a proteinaceous material is contacted under aqueous conditions with a proteolytic enzyme combination having an exopeptidase specific for peptides having a proline in the penultimate N-terminus. Optionally, the exopeptidase is specific for peptides having as an N-terminus a five amino acid sequence of X-Pro-Gln-Gln-Pro- wherein X is the amino terminal amino acid and can be any naturally occurring amino acid, Pro is proline and Gln is glutamine.

Optionally, the exopeptidase has a sequence having at least 70% sequence identity to one of MalPro11 (SEQ ID NO:1), MciPro4 (SEQ ID NO:2), TciPro1 (SEQ ID NO:3), FvePro4 (SEQ ID NO: 4), and SspPro2 (SEQ ID NO:5) or an active fragment thereof. Optionally, the exopeptidase has a sequence with at least 80% sequence identity to one of MalPro11 (SEQ ID NO:1), MciPro4 (SEQ ID NO:2), TciPro1 (SEQ ID NO:3), FvePro4 (SEQ ID NO: 4), and SspPro2 (SEQ ID NO:5) or an active fragment thereof. Optionally, the exopeptidase has a sequence with at least 85% sequence identity to one of MalPro11 (SEQ ID NO:1), MciPro4 (SEQ ID NO:2), TciPro1 (SEQ ID NO:3), FvePro4 (SEQ ID NO: 4), and SspPro2 (SEQ ID NO:5) or an active fragment thereof. Optionally, the exopeptidase has a sequence with at least 90% sequence identity to one of MalPro11 (SEQ ID NO:1), MciPro4 (SEQ ID NO:2), TciPro1 (SEQ ID NO:3), FvePro4 (SEQ ID NO: 4), and SspPro2 (SEQ ID NO:5) or an active fragment thereof.

Optionally, the exopeptidase has a sequence with at least 95% sequence identity to one of MalPro11 (SEQ ID NO:1), MciPro4 (SEQ ID NO:2), TciPro1 (SEQ ID NO:3), FvePro4 (SEQ ID NO: 4), and SspPro2 (SEQ ID NO:5) or an active fragment thereof. Optionally, the exopeptidase has a sequence with at least 99% sequence identity to one of MalPro11 (SEQ ID NO:1), MciPro4 (SEQ ID NO:2), TciPro1 (SEQ ID NO:3), FvePro4 (SEQ ID NO: 4), and SspPro2 (SEQ ID NO:5) or an active fragment thereof. Optionally, the exopeptidase has a sequence according to one of MalPro11 (SEQ ID NO:1), MciPro4 (SEQ ID NO:2), TciPro1 (SEQ ID NO:3), FvePro4 (SEQ ID NO: 4), and SspPro2 (SEQ ID NO:5) or an active fragment thereof. Optionally, the proteolytic enzyme mixture has a second exopeptidase. Preferably, the second exopeptidase is an aminopeptidase. Optionally, the aminopeptidase has a sequence with at least 70% sequence identity to one of SEQ ID NO:10, SEQ ID NO:11, SEQ ID NO:12, SEQ ID NO:13, SEQ ID NO:14, SEQ ID NO:15, SEQ ID NO:16, SEQ ID NO:17 and SEQ ID NO:28 or an aminopeptidase active fragment thereof. Optionally, the aminopeptidase has a sequence with at least 80% sequence identity to one of SEQ ID NO:10, SEQ ID NO:11, SEQ ID NO:12, SEQ ID NO:13, SEQ ID NO:14, SEQ ID NO:15, SEQ ID NO:16, SEQ ID NO:17 and SEQ ID NO:28 or an aminopeptidase active fragment thereof. Optionally, the aminopeptidase has a sequence with at least 85% sequence identity to one of SEQ ID NO:10, SEQ ID NO:11, SEQ ID NO:12, SEQ ID NO:13, SEQ ID NO:14, SEQ ID NO:15, SEQ ID NO:16, SEQ ID NO:17 and SEQ ID NO:28 or an aminopeptidase active fragment thereof. Optionally, the aminopeptidase has a sequence with at least 90% sequence identity to one of SEQ ID NO:10, SEQ ID NO:11, SEQ ID NO:12, SEQ ID NO:13, SEQ ID N0-14, SEQ ID NO:15, SEQ ID NO:16, SEQ ID NO:17 and SEQ ID NO:28 or an aminopeptidase active fragment thereof

Optionally, the aminopeptidase has a sequence with at least 95% sequence identity to one of SEQ ID NO:10, SEQ ID NO:11, SEQ ID NO:12, SEQ ID NO:13, SEQ ID NO:14, SEQ ID NO:15, SEQ ID NO:16, SEQ ID NO:17 and SEQ ID NO:28 or an aminopeptidase active fragment thereof. Optionally, the aminopeptidase has a sequence with at least 99% sequence identity to one of SEQ ID NO:10, SEQ ID NO:11, SEQ ID NO:12, SEQ ID NO:13, SEQ ID NO:14, SEQ ID NO:15, SEQ ID NO:16, SEQ ID NO:17 and SEQ ID NO:28 or an aminopeptidase active fragment thereof. Optionally, the aminopeptidase has a sequence according to one of SEQ ID NO:10. SEQ ID NO:11, SEQ ID NO:12, SEQ ID NO:13, SEQ ID NO:14, SEQ ID NO:15, SEQ ID NO:16, SEQ ID NO:17 and SEQ ID NO:28 or an aminopeptidase active fragment thereof. Optionally, the aminopeptidase has a sequence according to SEQ ID NO:10 or an aminopeptidase active fragment thereof.

Optionally, the proteolytic enzyme mixture also has an endopeptidase. Preferably, the endopeptidase has a sequence with at least 70% sequence identity to one of SEQ ID NO:18, SEQ ID NO:19, SEQ ID NO:20, SE ID NO:21, SEQ ID NO:22. SEQ ID NO:23, SEQ ID NO:24, SEQ ID NO:25 SEQ ID NO:26, and SEQ ID NO:27 or an endopeptidase active fragment thereof. Optionally, the endopeptidase has a sequence with at least 80% sequence identity to one of SEQ ID NO:18, SEQ ID NO:19, SEQ ID NO:20, SEQ ID NO:21, SEQ ID NO:22, SEQ ID NO:23, SEQ ID NO:24, SEQ ID NO:25 SEQ ID NO:26, and SEQ ID NO:27 or an endopeptidase active fragment thereof. Optionally, the endopeptidase has a sequence with at least 85% sequence identity to one of SEQ ID NO:18, SEQ ID NO:19, SEQ ID NO:20, SEQ ID NO:21, SEQ ID NO:22, SEQ ID NO:23, SEQ ID NO:24, SEQ ID NO:25 SEQ ID NO:26, and SEQ ID NO:27 or an endopeptidase active fragment thereof. Optionally, the endopeptidase has a sequence with at least 90% sequence identity to one of SEQ ID NO:18, SEQ ID NO:19. SEQ ID NO:20, SEQ ID NO:21, SEQ ID NO:22, SEQ ID NO:23, SEQ ID NO:24, SEQ ID NO:25 SEQ ID NO:26, and SEQ ID NO:27 or an endopeptidase active fragment thereof. Optionally, the endopeptidase has a sequence with at least 95% sequence identity to one of SEQ ID NO:18, SEQ ID NO:19, SEQ ID NO:20, SEQ ID NO:21, SEQ ID NO:22, SEQ ID NO:23, SEQ ID NO:24, SEQ ID NO:25 SEQ ID NO:26, and SEQ ID NO:27 or an endopeptidase active fragment thereof. Optionally, the endopeptidase has a sequence with at least 99% sequence identity to one of SEQ ID NO:18, SEQ ID NO:19, SEQ ID NO:20, SEQ ID NO:21, SEQ ID NO:22, SEQ ID NO:23, SEQ ID NO:24, SEQ 11) NO:25 SEQ ID NO:26, and SEQ 11) NO:27 or an endopeptidase active fragment thereof. Optionally, the endopeptidase has a sequence according to one of SEQ ID NO:18, SEQ ID NO:19. SEQ ID NO:20, SEQ ID NO:21, SEQ ID NO:22, SEQ ID NO:23, SEQ ID NO:24, SEQ ID NO:25 SEQ ID NO:26, and SEQ ID NO:27 or an endopeptidase active fragment thereof.

Optionally, the proteinaceous material is a vegetable derived protein, an animal derived protein, a fish derived protein, an insect derived protein or a microbial derived protein. Optionally, the proteinaceous material comprises gluten, soy protein, milk protein, egg protein, whey, casein, meat, hemoglobin or myosin.

Optionally, the proteolytic enzyme mixture has at least an exopeptidase specific for peptides having a proline in the penultimate N-terminus, a second exopeptidase and an endopeptidase as described above. Optionally, these enzymes are used to treat the proteinaceous material at the same time. Optionally, these enzymes are used at different times.

Optionally, the method for producing a protein hydrolysate is for producing hydrolysates having elevated levels of glutamic acid. Optionally, the proteolytic enzyme mixture has a glutaminase. Optionally, the glutaminase has a sequence with at least 70% sequence identity to SEQ ID NO:29 or a glutaminase active fragment thereof. Optionally, the glutaminase has a sequence with at least 80% sequence identity to SEQ ID NO:29 or a glutaminase active fragment thereof. Optionally, the glutaminase has a sequence with at least 85% sequence identity to SEQ ID NO:29 or a glutaminase active fragment thereof. Optionally, the glutaminase has a sequence with at least 90% sequence identity to SEQ ID NO:29 or a glutaninase active fragment thereof. Optionally, the glutaminase has a sequence with at least 95% sequence identity to SEQ ID NO:29 or a glutaminase active fragment thereof. Optionally, the glutaminase has a sequence with at least 99% sequence identity to SEQ ID NO:29 or a glutaminase active fragment thereof. Optionally, the glutaminase has a sequence according to SEQ ID NO:29 or a glutaminase active fragment thereof. According to this aspect of the present invention, the proteinaceous material is optionally gluten.

Optionally, the method for producing a protein hydrolysate is for producing hydrolysates having elevated levels of proline.

In other aspect of the present invention, a protein hydrolysate is presented produced according to any of the methods disclosed above.

In other aspect of the present invention, a food product is presented having a protein hydrolysate as described above.

### BRIEF DESCRIPTION OF THE BIOLOGICAL SEQUENCES

SEQ ID NO: 1 sets forth the protein sequence of full length MalPro11.

SEQ ID NO: 2 sets forth the protein sequence of full length MciPro4.

SEQ ID NO: 3 sets forth the protein sequence of full length TciPro1.

SEQ ID NO: 4 sets forth the protein sequence of full length FvePro4.

SEQ ID NO: 5 sets forth the protein sequence of full length SspPro2.

SEQ ID NO: 6 is the DNA sequence of the additional 5′ DNA fragment in pGXT-MalPro11, pGXT-MciPro4 and pGXT-TciPro1.

SEQ ID NO: 7 sets forth the protein sequence of predicted leader-truncated FvePro4.

SEQ ID NO: 8 sets forth the protein sequence of predicted leader-truncated SspPro2.

SEQ ID NO: 9 sets forth the protein sequence of the pentapeptide substrate.

SEQ ID NO:10 sets forth the protein sequence of predicted leader-truncated AcPepN2 Tri035.

SEQ ID NO:11 sets forth the protein sequence of predicted leader-truncated aminopeptidase Tr031.

SEQ ID NO:12 sets forth the protein sequence of predicted leader-truncated aminopeptidase Tr032.

SEQ ID NO:13 sets forth the protein sequence of predicted leader-truncated aminopeptidase Tr033.

SEQ ID NO:14 sets forth the protein sequence of predicted leader-truncated aminopeptidase Tr034.

SEQ ID NO:15 sets forth the protein sequence of predicted leader-truncated aminopeptidase Tr036.

SEQ ID NO:16 sets forth the protein sequence of predicted leader-truncated aminopeptidase Tr037.

SEQ ID NO:17 sets forth the protein sequence of predicted leader-truncated aminopeptidase Tr038.

SEQ ID NO:18 sets forth the protein sequence of mature Subtilisin A.

SEQ ID NO:19 sets forth the protein sequence of mature Subtilisin BPN′.

SEQ ID NO:20 sets forth the protein sequence of mature Subtilisin lentus.

SEQ ID NO:21 sets forth the protein sequence of mature Thermolysin.

SEQ ID NO:22 sets forth the protein sequence of mature Bacillolysin.

SEQ ID NO:23 sets forth the protein sequence of mature Trichodermapepsin.

SEQ ID NO:23 sets forth the protein sequence of mature Trichodermapepsin.

SEQ ID NO:24 sets forth the protein sequence of mature Bromealin.

SEQ ID NO:25 sets forth the protein sequence of mature Aspergillopepsin.

SEQ ID NO:26 sets forth the protein sequence of mature Trypsin 1.

SEQ ID NO:27 sets forth the protein sequence of mature Chymotrypsin A.

SEQ ID NO:28 sets forth the protein sequence of predicted leader-truncated aminopeptidase Tr063.

SEQ ID NO:29 sets forth the protein sequence of the full length glutaminase.

### DESCRIPTION OF FIGURES

FIG. 3A. depicts dose response curves of purified MalPro11, MciPro4, TciPro1, FvePro4 and SspPro2 on Phe-Pro.

FIG. 3B. depicts dose response curves of purified MalPro11, MciPro4, TciPro1, FvePro4 and SspPro2 on Ser-Pro.

FIG. 4. depicts the pH profiles of purified MalPro11, MciPro4, TciPro1, FvePro4 and SspPro2.

FIG. 5. depicts the temperature profiles of purified MalPro11, MciPro4, TciPro1, FvePro4 and SspPro2.

FIG. 6. depicts the thermostability tests of purified MalPro11, MciPro4, TciPro1, FvePro4 and SspPro2.

FIG. 7. depicts Gln-Pro-Gln-Gln-Pro hydrolysis analyses of purified MalPro11, MciPro4, TciPro1, FvePro4 and SspPro2.

FIG. 8. shows the effect of different doses of SspPro2 on free glutamic acid formation from gluten pre-hydrolysate after 19 h incubation together with AcPepN2 and glutaminase. Reference: Contains gluten pre-hydrolysate+glutaminase. AcPepN2 contains gluten pre-hydrolysate+glutaminase+AcPepN2. The two last samples contain the same as AcPepN2 but with additionally 131 μg or 392 μg pr. mL pre-hydrolysate.

FIG. 9. is the same as FIG. 8 but after 26 h of incubation.

FIG. 10. shows the effect of different X-ProAP's on glutamic acid yield. Incubation 24 h at 50° C. with pre-hydrolysate, glutaminase and mentioned enzymes. Dose of X-ProAP is in all cases 312 μg/mL of pre-hydrolysate.

FIG. 11 shows the effect of AoX-ProAP and HX-ProAP on glutamic acid yield. Incubation 42 h at 50° C. with pre-hydrolysate, glutaminase and mentioned enzymes. Dose of X-ProAP's is 15 μg/mL of pre-hydrolysate.

FIG. 12 shows overlaid chromatograms of hydrolysates. Solid line: 26 h incubation of pre-hydrolysate with glutaminase and AcPepN2. Dashed line 26 h incubation of pre-hydrolysate with glutaminase, AcPepN2 and SspPro2. The time intervals where amino acids (AA's) primarily elute and the interval where DP2 to DP5 primarily elute are indicated on the figure.

FIG. 13 shows overlaid chromatograms of hydrolysates. Solid line: 26 h incubation of pre-hydrolysate with glutaminase and AcPepN2. Dashed line 26 h incubation of pre-hydrolysate with glutaminase, AcPepN2 and HX-ProAP. The time intervals where amino acids (AA's) primarily elute and the interval where DP2 to DP5 primarily elute are indicated on the figure

### DETAILED DESCRIPTION OF THE INVENTION

The practice of the present teachings will employ, unless otherwise indicated, conventional techniques of molecular biology (including recombinant techniques), microbiology, cell biology, and biochemistry, which are within the skill of the art. Such techniques are explained fully in the literature, for example, Molecular Cloning: A Laboratory Manual, second edition (Sambrook et al., 1989); Oligonucleotide Synthesis (M. J. Gait, ed., 1984; Current Protocols in Molecular Biology (F. M. Ausubel et al., eds., 1994), PCR: The Polymerase Chain Reaction (Mullis et al., eds., 1994); Gene Transfer and Expression: A Laboratory Manual (Kriegler, 1990), and The Alcohol Textbook (Ingledew et al., eds., Fifth Edition, 2009), and Essentials of Carbohydrate Chemistry and Biochemistry (Lindhorste, 2007).

Unless defined otherwise herein, all technical and scientific terms used herein have the same meaning as commonly understood by one of ordinary skill in the art to which the present teachings belong. Singleton, et al., Dictionary of Microbiology and Molecular Biology, second ed., John Wiley and Sons, New York (1994), and Hale & Markham, The Harper Collins Dictionary of Biology, Harper Perennial, NY (1991) provide one of skill with a general dictionary of many of the terms used in this invention. Any methods and materials similar or equivalent to those described herein can be used in the practice or testing of the present teachings.

Numeric ranges provided herein are inclusive of the numbers defining the range.

### Definitions

The terms, “wild-type,” “parental,” or “reference,” with respect to a polypeptide, refer to a naturally-occurring polypeptide that does not include a man-made substitution, insertion, or deletion at one or more amino acid positions. Similarly, the terms “wild-type,” “parental,” or “reference,” with respect to a polynucleotide, refer to a naturally-occurring polynucleotide that does not include a man-made nucleoside change. However, note that a polynucleotide encoding a wild-type, parental, or reference polypeptide is not limited to a naturally-occurring polynucleotide, and encompasses any polynucleotide encoding the wild-type, parental, or reference polypeptide.

Reference to the wild-type polypeptide is understood to include the mature form of the polypeptide. A “mature” polypeptide or variant, thereof, is one in which a signal sequence is absent, for example, cleaved from an immature form of the polypeptide during or following expression of the polypeptide.

The term “variant,” with respect to a polypeptide, refers to a polypeptide that differs from a specified wild-type, parental, or reference polypeptide in that it includes one or more naturally-occurring or man-made substitutions, insertions, or deletions of an amino acid. Similarly, the term “variant,” with respect to a polynucleotide, refers to a polynucleotide that differs in nucleotide sequence from a specified wild-type, parental, or reference polynucleotide. The identity of the wild-type, parental, or reference polypeptide or polynucleotide will be apparent from context.

The term “recombinant,” when used in reference to a subject cell, nucleic acid, protein or vector, indicates that the subject has been modified from its native state. Thus, for example, recombinant cells express genes that are not found within the native (non-recombinant) form of the cell, or express native genes at different levels or under different conditions than found in nature. Recombinant nucleic acids differ from a native sequence by one or more nucleotides and/or are operably linked to heterologous sequences, e.g., a heterologous promoter in an expression vector. Recombinant proteins may differ from a native sequence by one or more amino acids and/or are fused with heterologous sequences. A vector comprising a nucleic acid encoding a protease is a recombinant vector.

The terms “recovered,” “isolated,” and “separated,” refer to a compound, protein (polypeptides), cell, nucleic acid, amino acid, or other specified material or component that is removed from at least one other material or component with which it is naturally associated as found in nature. An “isolated” polypeptides, thereof, includes, but is not limited to, a culture broth containing secreted polypeptide expressed in a heterologous host cell.

The term “purified” refers to material (e.g., an isolated polypeptide or polynucleotide) that is in a relatively pure state, e.g., at least about 90% pure, at least about 95% pure, at least about 98% pure, or even at least about 99% pure.

The term “enriched” refers to material (e.g., an isolated polypeptide or polynucleotide) that is in about 50% pure, at least about 60% pure, at least about 70% pure, or even at least about 70% pure.

A “pH range,” with reference to an enzyme, refers to the range of pH values under which the enzyme exhibits catalytic activity.

The terms “pH stable” and “pH stability,” with reference to an enzyme, relate to the ability of the enzyme to retain activity over a wide range of pH values for a predetermined period of time (e.g., 15 min., 30 min., 1 hour).

The term “amino acid sequence” is synonymous with the terms “polypeptide,” “protein,” and “peptide,” and are used interchangeably. Where such amino acid sequences exhibit activity, they may be referred to as an “enzyme.” The conventional one-letter or three-letter codes for amino acid residues are used, with amino acid sequences being presented in the standard amino-to-carboxy terminal orientation (i.e., N→C).

The term “nucleic acid” encompasses DNA, RNA, heteroduplexes, and synthetic molecules capable of encoding a polypeptide. Nucleic acids may be single stranded or double stranded, and may be chemical modifications. The terms “nucleic acid” and “polynucleotide” are used interchangeably. Because the genetic code is degenerate, more than one codon may be used to encode a particular amino acid, and the present compositions and methods encompass nucleotide sequences that encode a particular amino acid sequence. Unless otherwise indicated, nucleic acid sequences are presented in 5′-to-3′ orientation.

“Hybridization” refers to the process by which one strand of nucleic acid forms a duplex with, i.e., base pairs with, a complementary strand, as occurs during blot hybridization techniques and PCR techniques. Stringent hybridization conditions are exemplified by hybridization under the following conditions: 65° C. and 0.1×SSC (where 1×SSC=0.15 M NaCl, 0.015 M Na₃ citrate, pH 7.0). Hybridized, duplex nucleic acids are characterized by a melting temperature (Tₘ), where one half of the hybridized nucleic acids are unpaired with the complementary strand. Mismatched nucleotides within the duplex lower the Tₘ. Very stringent hybridization conditions involve 68° C. and 0.1×SSC

A “synthetic” molecule is produced by in vitro chemical or enzymatic synthesis rather than by an organism.

The terms “transformed,” “stably transformed,” and “transgenic,” used with reference to a cell means that the cell contains a non-native (e.g., heterologous) nucleic acid sequence integrated into its genome or carried as an episome that is maintained through multiple generations.

The term “introduced” in the context of inserting a nucleic acid sequence into a cell, means “transfection”, “transformation” or “transduction,” as known in the art.

A “host strain” or “host cell” is an organism into which an expression vector, phage, virus, or other DNA construct, including a polynucleotide encoding a polypeptide of interest (e.g., a protease) has been introduced. Exemplary host strains are microorganism cells (e.g., bacteria, filamentous fungi, and yeast) capable of expressing the polypeptide of interest. The term “host cell” includes protoplasts created from cells.

The term “heterologous” with reference to a polynucleotide or protein refers to a polynucleotide or protein that does not naturally occur in a host cell.

The term “endogenous” with reference to a polynucleotide or protein refers to a polynucleotide or protein that occurs naturally in the host cell.

The term “expression” refers to the process by which a polypeptide is produced based on a nucleic acid sequence. The process includes both transcription and translation.

A “selective marker” or “selectable marker” refers to a gene capable of being expressed in a host to facilitate selection of host cells carrying the gene. Examples of selectable markers include but are not limited to antimicrobials (e.g., hygromycin, bleomycin, or chloramphenicol) and/or genes that confer a metabolic advantage, such as a nutritional advantage on the host cell.

A “vector” refers to a polynucleotide sequence designed to introduce nucleic acids into one or more cell types. Vectors include cloning vectors, expression vectors, shuttle vectors, plasmids, phage particles, cassettes and the like.

An “expression vector” refers to a DNA construct comprising a DNA sequence encoding a polypeptide of interest, which coding sequence is operably linked to a suitable control sequence capable of effecting expression of the DNA in a suitable host. Such control sequences may include a promoter to effect transcription, an optional operator sequence to control transcription, a sequence encoding suitable ribosome binding sites on the mRNA, enhancers and sequences which control termination of transcription and translation.

The term “operably linked” means that specified components are in a relationship (including but not limited to juxtaposition) permitting them to function in an intended manner. For example, a regulatory sequence is operably linked to a coding sequence such that expression of the coding sequence is under control of the regulatory sequences.

A “signal sequence” is a sequence of amino acids attached to the N-terminal portion of a protein, which facilitates the secretion of the protein outside the cell. The mature form of an extracellular protein lacks the signal sequence, which is cleaved off during the secretion process.

“Biologically active” refers to a sequence having a specified biological activity, such an enzymatic activity.

The term “specific activity” refers to the number of moles of substrate that can be converted to product by an enzyme or enzyme preparation per unit time under specific conditions. Specific activity is generally expressed as units (U)/mg of protein.

As used herein, “percent sequence identity” means that a particular sequence has at least a certain percentage of amino acid residues identical to those in a specified reference sequence, when aligned using the CLUSTAL W algorithm with default parameters. See Thompson et al. (1994) Nucleic Acids Res. 22:4673-4680. Default parameters for the CLUSTAL W algorithm are:

| | |
|---|---|
| Gap opening penalty: | 10.0 |
| Gap extension penalty: | 0.05 |
| Protein weight matrix: | BLOSUM series |
| DNA weight matrix: | IUB |
| Delay divergent sequences %: | 40 |
| Gap separation distance: | 8 |
| DNA transitions weight: | 0.50 |
| List hydrophilic residues: | GPSNDQEKR |
| Use negative matrix: | OFF |
| Toggle Residue specific penalties: | ON |
| Toggle hydrophilic penalties: | ON |
| Toggle end gap separation penalty | OFF. |

Deletions are counted as non-identical residues, compared to a reference sequence. Deletions occurring at either terminus are included. For example, a variant with five amino acid deletions of the C-terminus of the mature 617 residue polypeptide would have a percent sequence identity of 99% (612/617 identical residues×100, rounded to the nearest whole number) relative to the mature polypeptide. Such a variant would be encompassed by a variant having “at least 99% sequence identity” to a mature polypeptide.

“Fused” polypeptide sequences are connected, i.e., operably linked, via a peptide bond between two subject polypeptide sequences.

The term “filamentous fungi” refers to all filamentous forms of the subdivision Eumycotina, particularly Pezizomycotina species.

The term “about” refers to ±5% to the referenced value.

The terms “peptidase” or “protease” refer to enzymes that hydrolyzes peptide bonds in a poly or oligo peptide. As used herein, the terms peptidase or protease include the enzymes assigned to subclass EC 3.4.

The terms “exopeptidase” or “exoprotease” refer to peptidases that act to hydrolyze peptide bonds at the ends (amino or carboxyl) of a poly or oligopeptide. Exopeptidases that act at the amino terminus of a polypeptide are referred to herein as aminopeptidases. Aminopeptidases can act to cleave or liberate single amino acids, dipeptides and tripeptides from the amino terminus depending on their specificity. Exopeptidases that act at the carboxy terminus are referred to herein as carboxypepitdases. Carboxypeptidases can act to cleave or liberate single amino acids, dipeptides and tripeptides from the carboxy terminus depending on their specificity.

The term “endopeptidase” or “endoprotease” refers to a peptidase or protease the hydrolyzes internal peptide bonds in a protein or oligo peptide

A “hydrolysate” is a product of a reaction wherein a compound is cleaved with water. Hydrolysates of protein or “protein hydrolysates” occur when protein bonds are hydrolyzed with water. Hydrolysis of proteins may be increased by heat or enzymes. During hydrolysis proteins are broken down into smaller proteins, peptides and free amino acids.

Other definitions are set forth below.

### Additional Mutations

In some embodiments, the present proteases further include one or more mutations that provide a further performance or stability benefit. Exemplary performance benefits include but are not limited to increased thermal stability, increased storage stability, increased solubility, an altered pH profile, increased specific activity, modified substrate specificity, modified substrate binding, modified pH-dependent activity, modified pH-dependent stability, increased oxidative stability, and increased expression. In some cases, the performance benefit is realized at a relatively low temperature. In some cases, the performance benefit is realized at relatively high temperature.

Furthermore, the present proteases may include any number of conservative amino acid substitutions. Exemplary conservative amino acid substitutions are listed in the following Table.

TABLE 1
Conservative amino acid substitutions

| For Amino | | |
|---|---|---|
| Acid | Code | Replace with any of |
| Alanine | A | D-Ala, Gly, beta-Ala, L-Cys, D-Cys |
| Arginine | R | D-Arg, Lys, D-Lys, homo-Arg, D-homo-Arg, Met, |
| | | Ile, D-Met, D-Ile, Orn, D-Orn |
| Asparagine | N | D-Asn, Asp, D-Asp, Glu, D-Glu, Gln, D-Gln |
| Aspartic | D | D-Asp, D-Asn, Asn, Glu, D-Glu, Gln, D-Gln |
| Acid | | |
| Cysteine | C | D-Cys, S-Me-Cys, Met, D-Met, Thr, D-Thr |
| Glutamine | Q | D-Gln, Asn, D-Asn, Glu, D-Glu, Asp, D-Asp |
| Glutamic | E | D-Glu, D-Asp, Asp, Asn, D-Asn, Gln, D-Gln |
| Acid | | |
| Glycine | G | Ala, D-Ala, Pro, D-Pro, b-Ala, Acp |
| Isoleucine | I | D-Ile, Val, D-Val, Leu, D-Leu, Met, D-Met |
| Leucine | L | D-Leu, Val, D-Val, Leu, D-Leu, Met, D-Met |
| Lysine | K | D-Lys, Arg, D-Arg, homo-Arg, D-homo-Arg, Met, |
| | | D-Met, Ile, D-Ile, Orn, D-Orn |
| Methionine | M | D-Met, S-Me-Cys, Ile, D-Ile, Leu, D-Leu, Val, D- |
| | | Val |
| Phenylalanine | F | D-Phe, Tyr, D-Thr, L-Dopa, His, D-His, Trp, D- |
| | | Trp, Trans-3,4, or 5-phenylproline, cis-3,4, |
| | | or 5-phenylproline |
| Proline | P | D-Pro, L-I-thioazolidine-4-carboxylic acid, D-or |
| | | L-1-oxazolidine-4-carboxylic acid |
| Serine | S | D-Ser, Thr, D-Thr, allo-Thr, Met, D-Met, Met(O), |
| | | D-Met(O), L-Cys, D-Cys |
| Threonine | T | D-Thr, Ser, D-Ser, allo-Thr, Met, |
| | | D-Met, Met(O), D-Met(O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met |

The reader will appreciate that some of the above mentioned conservative mutations can be produced by genetic manipulation, while others are produced by introducing synthetic amino acids into a polypeptide by genetic or other means.

The present protease may be “precursor,” “immature,” or “full-length,” in which case they include a signal sequence, or “mature,” in which case they lack a signal sequence. Mature forms of the polypeptides are generally the most useful. Unless otherwise noted, the amino acid residue numbering used herein refers to the mature forms of the respective protease polypeptides. The present protease polypeptides may also be truncated to remove the N or C-termini, so long as the resulting polypeptides retain protease activity. In addition, protease enzymes may be active fragments derived from a longer amino acid sequence. Active fragments are characterized by retaining some or all of the activity of the full length enzyme but have deletions from the N-terminus, from the C-terminus or internally or combinations thereof.

The present protease may be a “chimeric” or “hybrid” polypeptide, in that it includes at least a portion of a first protease polypeptide, and at least a portion of a second protease polypeptide. The present protease may further include heterologous signal sequence, an epitope to allow tracking or purification, or the like. Exemplary heterologous signal sequences are from B. licheniformis amylase (LAT), B. subtilis (AmyE or AprE), and Streptomyces CelA.

### Production of Variant Proteases

The present protease can be produced in host cells, for example, by secretion or intracellular expression. A cultured cell material (e.g., a whole-cell broth) comprising a protease can be obtained following secretion of the protease into the cell medium. Optionally, the protease can be isolated from the host cells, or even isolated from the cell broth, depending on the desired purity of the final protease. A gene encoding a protease can be cloned and expressed according to methods well known in the art. Suitable host cells include bacterial, fungal (including yeast and filamentous fungi), and plant cells (including algae). Particularly useful host cells include Aspergillus niger, Aspergillus oryzae or Trichoderma reesei. Other host cells include bacterial cells, e.g., Bacillus subtilis or B. licheniformis, as well as Streptomyces, E Coli.

The host cell further may express a nucleic acid encoding a homologous or heterologous protease, i.e., a protease that is not the same species as the host cell, or one or more other enzymes. The protease may be a variant protease. Additionally, the host may express one or more accessory enzymes, proteins, peptides.

### Vectors

A DNA construct comprising a nucleic acid encoding a protease can be constructed to be expressed in a host cell. Because of the well-known degeneracy in the genetic code, variant polynucleotides that encode an identical amino acid sequence can be designed and made with routine skill. It is also well-known in the art to optimize codon use for a particular host cell. Nucleic acids encoding protease can be incorporated into a vector. Vectors can be transferred to a host cell using well-known transformation techniques, such as those disclosed below.

The vector may be any vector that can be transformed into and replicated within a host cell. For example, a vector comprising a nucleic acid encoding a protease can be transformed and replicated in a bacterial host cell as a means of propagating and amplifying the vector. The vector also may be transformed into an expression host, so that the encoding nucleic acids can be expressed as a functional protease. Host cells that serve as expression hosts can include filamentous fungi, for example. The Fungal Genetics Stock Center (FGSC) Catalogue of Strains lists suitable vectors for expression in fungal host cells. See FGSC, Catalogue of Strains, University of Missouri, at www.fgsc.net (last modified Jan. 17, 2007). A representative vector is pJG153, a promoterless Cre expression vector that can be replicated in a bacterial host. See Harrison et al. (June 2011) Applied Environ. Microbiol. 77: 3916-22. pJG153 can be modified with routine skill to comprise and express a nucleic acid encoding a protease.

A nucleic acid encoding a protease can be operably linked to a suitable promoter, which allows transcription in the host cell. The promoter may be any DNA sequence that shows transcriptional activity in the host cell of choice and may be derived from genes encoding proteins either homologous or heterologous to the host cell. Exemplary promoters for directing the transcription of the DNA sequence encoding a protease, especially in a bacterial host, are the promoter of the lac operon of E. coli, the Streptomyces coelicolor agarase gene dagA or celA promoters, the promoters of the Bacillus licheniformis α-amylase gene (amyL), the promoters of the Bacillus stearothermophilus maltogenic amylase gene (amyM), the promoters of the Bacillus amvloliquefaciens α-amylase (amyQ), the promoters of the Bacillus subtilis xylA and xylB genes etc. For transcription in a fungal host, examples of useful promoters are those derived from the gene encoding Aspergillus oryzae TAKA amylase, Rhizomucor miehei aspartic proteinase, Aspergillus niger neutral α-amylase, A. niger acid stable α-amylase, A. niger glucoamylase, Rhizomucor miehei lipase, A. oryzae alkaline protease, A. oryzae triose phosphate isomerase, or A. nidulans acetamidase. When a gene encoding a protease is expressed in a bacterial species such as E. coli, a suitable promoter can be selected, for example, from a bacteriophage promoter including a T7 promoter and a phage lambda promoter. Examples of suitable promoters for the expression in a yeast species include but are not limited to the Gal 1 and Gal 10 promoters of Saccharomyces cerevisiae and the Pichia pastoris AOX1 or AOX2 promoters. cbh1 is an endogenous, inducible promoter from T. reesei. See Liu et al. (2008) “Improved heterologous gene expression in Trichoderma reesei by cellobiohydrolase I gene (cbh1) promoter optimization,” Acta Biochim. Biophys. Sin (Shanghai) 40(2): 158-65.

The coding sequence can be operably linked to a signal sequence. The DNA encoding the signal sequence may be the DNA sequence naturally associated with the protease gene to be expressed or from a different Genus or species. A signal sequence and a promoter sequence comprising a DNA construct or vector can be introduced into a fungal host cell and can be derived from the same source. For example, the signal sequence is the cbh1 signal sequence that is operably linked to a cbh1 promoter.

An expression vector may also comprise a suitable transcription terminator and, in eukaryotes, polyadenylation sequences operably linked to the DNA sequence encoding a variant protease. Termination and polyadenylation sequences may suitably be derived from the same sources as the promoter.

The vector may further comprise a DNA sequence enabling the vector to replicate in the host cell. Examples of such sequences are the origins of replication of plasmids pUC19, pACYC177, pUB110, pE194, pAMB1, and pIJ702.

The vector may also comprise a selectable marker, e.g., a gene the product of which complements a defect in the isolated host cell, such as the dal genes from B. subtilis or B. licheniformis, or a gene that confers antibiotic resistance such as, e.g., ampicillin, kanamycin, chloramphenicol or tetracycline resistance. Furthermore, the vector may comprise Aspergillus selection markers such as amdS, argB, niaD and xxsC, a marker giving rise to hygromycin resistance, or the selection may be accomplished by co-transformation, such as known in the art. See e.g., International PCT Application WO 91/17243.

Intracellular expression may be advantageous in some respects, e.g., when using certain bacteria or fungi as host cells to produce large amounts of protease for subsequent enrichment or purification. Extracellular secretion of protease into the culture medium can also be used to make a cultured cell material comprising the isolated protease.

The expression vector typically includes the components of a cloning vector, such as, for example, an element that permits autonomous replication of the vector in the selected host organism and one or more phenotypically detectable markers for selection purposes. The expression vector normally comprises control nucleotide sequences such as a promoter, operator, ribosome binding site, translation initiation signal and optionally, a repressor gene or one or more activator genes. Additionally, the expression vector may comprise a sequence coding for an amino acid sequence capable of targeting the protease to a host cell organelle such as a peroxisome, or to a particular host cell compartment. Such a targeting sequence includes but is not limited to the sequence, SKL. For expression under the direction of control sequences, the nucleic acid sequence of the protease is operably linked to the control sequences in proper manner with respect to expression.

The procedures used to ligate the DNA construct encoding a protease, the promoter, terminator and other elements, respectively, and to insert them into suitable vectors containing the information necessary for replication, are well known to persons skilled in the art (see, e.g., Sambrook et al., MOLECULAR CLONING: A LABORATORY MANUAL, 2^{nd} ed., Cold Spring Harbor, 1989, and 3d ed., 2001).

### Transformation and Culture of Host Cells

An isolated cell, either comprising a DNA construct or an expression vector, is advantageously used as a host cell in the recombinant production of a protease. The cell may be transformed with the DNA construct encoding the enzyme, conveniently by integrating the DNA construct (in one or more copies) in the host chromosome. This integration is generally considered to be an advantage, as the DNA sequence is more likely to be stably maintained in the cell. Integration of the DNA constructs into the host chromosome may be performed according to conventional methods, e.g., by homologous or heterologous recombination. Alternatively, the cell may be transformed with an expression vector as described above in connection with the different types of host cells.

Examples of suitable bacterial host organisms are Gram positive bacterial species such as Bacillaceae including Bacillus subtilis, Bacillus licheniformis, Bacillus lentus, Bacillus brevis, Geobacillus (formerly Bacillus) stearothermophilus, Bacillus alkalophilus, Bacillus amvloliquefaciens, Bacillus coagulans, Bacillus lautus, Bacillus megaterium, and Bacillus thuringiensis; Streptomyces species such as Streptomyces murinus; lactic acid bacterial species including Lactococcus sp. such as Lactococcus lactis; Lactobacillus sp. including Lactobacillus reuteri; Leuconostoc sp.; Pediococcus sp.; and Streptococcus sp. Alternatively, strains of a Gram negative bacterial species belonging to Enterobacteriaceae including E. coli, or to Pseudomonadaceae can be selected as the host organism.

A suitable yeast host organism can be selected from the biotechnologically relevant yeasts species such as but not limited to yeast species such as Pichia sp., Hansenula sp., or Kluyveromyces, Yarrowinia, Schizosaccharomyces species or a species of Saccharomyces, including, Saccharomyces cerevisiae or a species belonging to Schizosaccharomyces such as, for example, S. pombe species. A strain of the methylotrophic yeast species, Pichia pastoris, can be used as the host organism. Alternatively, the host organism can be a Hansenula species. Suitable host organisms among filamentous fungi include species of Aspergillus, e.g., Aspergillus niger, Aspergillus oryzae, Aspergillus tubigensis, Aspergillus awamori, or Aspergillus nidulans. Alternatively, strains of a Fusarium species, e.g., Fusarium oxysporum or of a Rhizomucor species such as Rhizomucor miehei can be used as the host organism. Other suitable strains include Thermomyces and Mucor species. In addition, Trichoderma sp. can be used as a host. A suitable procedure for transformation of Aspergillus host cells includes, for example, that described in EP 238023. A protease expressed by a fungal host cell can be glycosylated, i.e., will comprise a glycosyl moiety. The glycosylation pattern can be the same or different as present in the wild-type protease. The type and/or degree of glycosylation may impart changes in enzymatic and/or biochemical properties.

It is advantageous to delete genes from expression hosts, where the gene deficiency can be cured by the transformed expression vector. Known methods may be used to obtain a fungal host cell having one or more inactivated genes. Gene inactivation may be accomplished by complete or partial deletion, by insertional inactivation or by any other means that renders a gene nonfunctional for its intended purpose, such that the gene is prevented from expression of a functional protein. Any gene from a Trichoderma sp. or other filamentous fungal host that has been cloned can be deleted, for example, cbh1, cbh2, egl1, and egl2 genes. Gene deletion may be accomplished by inserting a form of the desired gene to be inactivated into a plasmid by methods known in the art.

Introduction of a DNA construct or vector into a host cell includes techniques such as transformation; electroporation; nuclear microinjection; transduction; transfection, e.g., lipofection mediated and DEAE-Dextrin mediated transfection; incubation with calcium phosphate DNA precipitate; high velocity bombardment with DNA-coated microprojectiles; and protoplast fusion. General transformation techniques are known in the art. See, e.g., Sambrook et al. (2001), supra. The expression of heterologous protein in Trichoderma is described, for example, in U.S. Pat. No. 6,022,725. Reference is also made to Cao et al. (2000) Science 9:991-1001 for transformation of Aspergillus strains. Genetically stable transformants can be constructed with vector systems whereby the nucleic acid encoding a protease is stably integrated into a host cell chromosome. Transformants are then selected and purified by known techniques.

The preparation of Trichoderma sp. for transformation, for example, may involve the preparation of protoplasts from fungal mycelia. See Campbell et al. (1989) Curr. Genet. 16: 53-56. The mycelia can be obtained from germinated vegetative spores. The mycelia are treated with an enzyme that digests the cell wall, resulting in protoplasts. The protoplasts are protected by the presence of an osmotic stabilizer in the suspending medium. These stabilizers include sorbitol, mannitol, potassium chloride, magnesium sulfate, and the like. Usually the concentration of these stabilizers varies between 0.8 M and 1.2 M, e.g., a 1.2 M solution of sorbitol can be used in the suspension medium.

Uptake of DNA into the host Trichoderma sp. strain depends upon the calcium ion concentration. Generally, between about 10-50 mM CaCl₂ is used in an uptake solution. Additional suitable compounds include a buffering system, such as TE buffer (10 mM Tris, pH 7.4; 1 mM EDTA) or 10 mM MOPS, pH 6.0 and polyethylene glycol. The polyethylene glycol is believed to fuse the cell membranes, thus permitting the contents of the medium to be delivered into the cytoplasm of the Trichoderma sp. strain. This fusion frequently leaves multiple copies of the plasmid DNA integrated into the host chromosome.

Usually transformation of Trichoderma sp. uses protoplasts or cells that have been subjected to a permeability treatment, typically at a density of 105 to 107/mL, particularly 2×10⁶/mL. A volume of 100 μL of these protoplasts or cells in an appropriate solution (e.g., 1.2 M sorbitol and 50 mM CaCl₂) may be mixed with the desired DNA. Generally, a high concentration of PEG is added to the uptake solution. From 0.1 to 1 volume of 25% PEG 4000 can be added to the protoplast suspension; however, it is useful to add about 0.25 volumes to the protoplast suspension. Additives, such as dimethyl sulfoxide, heparin, spermidine, potassium chloride and the like, may also be added to the uptake solution to facilitate transformation. Similar procedures are available for other fungal host cells. See, e.g., U.S. Pat. No. 6,022,725.

### Expression

A method of producing a protease may comprise cultivating a host cell as described above under conditions conducive to the production of the enzyme and recovering the enzyme from the cells and/or culture medium.

The medium used to cultivate the cells may be any conventional medium suitable for growing the host cell in question and obtaining expression of a protease. Suitable media and media components are available from commercial suppliers or may be prepared according to published recipes (e.g., as described in catalogues of the American Type Culture Collection).

An enzyme secreted from the host cells can be used in a whole broth preparation. In the present methods, the preparation of a spent whole fermentation broth of a recombinant microorganism can be achieved using any cultivation method known in the art resulting in the expression of a protease. Fermentation may, therefore, be understood as comprising shake flask cultivation, small- or large-scale fermentation (including continuous, batch, fed-batch, or solid state fermentations) in laboratory or industrial fermenters performed in a suitable medium and under conditions allowing the protease to be expressed or isolated. The term “spent whole fermentation broth” is defined herein as unfractionated contents of fermentation material that includes culture medium, extracellular proteins (e.g., enzymes), and cellular biomass. It is understood that the term “spent whole fermentation broth” also encompasses cellular biomass that has been lysed or permeabilized using methods well known in the art.

An enzyme secreted from the host cells may conveniently be recovered from the culture medium by well-known procedures, including separating the cells from the medium by centrifugation or filtration, and precipitating proteinaceous components of the medium by means of a salt such as ammonium sulfate, followed by the use of chromatographic procedures such as ion exchange chromatography, affinity chromatography, or the like.

The polynucleotide encoding a protease in a vector can be operably linked to a control sequence that is capable of providing for the expression of the coding sequence by the host cell, i.e. the vector is an expression vector. The control sequences may be modified, for example by the addition of further transcriptional regulatory elements to make the level of transcription directed by the control sequences more responsive to transcriptional modulators. The control sequences may in particular comprise promoters.

Host cells may be cultured under suitable conditions that allow expression of a protease. Expression of the enzymes may be constitutive such that they are continually produced, or inducible, requiring a stimulus to initiate expression. In the case of inducible expression, protein production can be initiated when required by, for example, addition of an inducer substance to the culture medium, for example dexamethasone or IPTG or Sophorose. Polypeptides can also be produced recombinantly in an in vitro cell-free system, such as the TNT™ (Promega) rabbit reticulocyte system.

An expression host also can be cultured in the appropriate medium for the host, under aerobic conditions. Shaking or a combination of agitation and aeration can be provided, with production occurring at the appropriate temperature for that host, e.g., from about 25° C. to about 75° C. (e.g., 30° C. to 45° C.), depending on the needs of the host and production of the desired protease. Culturing can occur from about 12 to about 100 hours or greater (and any hour value there between, e.g., from 24 to 72 hours). Typically, the culture broth is at a pH of about 4.0 to about 8.0, again depending on the culture conditions needed for the host relative to production of a protease.

### Methods for Enriching and Purifying Proteases

Fermentation, separation, and concentration techniques are well known in the art and conventional methods can be used in order to prepare a protease polypeptide-containing solution.

After fermentation, a fermentation broth is obtained, the microbial cells and various suspended solids, including residual raw fermentation materials, are removed by conventional separation techniques in order to obtain a protease solution. Filtration, centrifugation, microfiltration, rotary vacuum drum filtration, ultrafiltration, centrifugation followed by ultra-filtration, extraction, or chromatography, or the like, are generally used.

It is desirable to concentrate a protease polypeptide-containing solution in order to optimize recovery. Use of unconcentrated solutions requires increased incubation time in order to collect the enriched or purified enzyme precipitate.

The enzyme containing solution is concentrated using conventional concentration techniques until the desired enzyme level is obtained. Concentration of the enzyme containing solution may be achieved by any of the techniques discussed herein. Exemplary methods of enrichment and purification include but are not limited to rotary vacuum filtration and/or ultrafiltration.

The enzyme solution is concentrated into a concentrated enzyme solution until the enzyme activity of the concentrated protease polypeptide-containing solution is at a desired level.

Enriched or purified enzymes can be made into a final product that is either liquid (solution, slurry) or solid (granular, powder).

### PREFERRED EMBODIMENTS OF THE INVENTION

In accordance with an aspect of the present invention, it was discovered that some aminopeptidases stall at or only slowly digest peptides or proteins having proline in the penultimate N-terminal position. In particular, it was discovered that these aminopeptidases will not digest proteins of peptides having the N-terminal sequence X-Pro-Gln-Gln-Pro- (where X is any amino acid). Use of such aminopeptidases in producing protein hydrolysates will result in a hydrolysate having low amounts of the X amino acid because of the resistance of such a peptide to digestion.

Glutamic acid in the form of mono sodium glutamate (MSG) is a commonly used flavor enhancer. It is responsible for savory or umami taste. MSG can be produced by enzymatic hydrolysis of protein. In this regard, gluten is high in glutamine and can be a source of MSG (glutamine can be converted to glutamic acid using glutaminase). In accordance with an aspect of the present invention, it was discovered that gluten contains significant amounts of the sequence X-Pro-Gln-Gln-Pro-, greatly limiting the amount of glutamine that can be liberated from the gluten.

In accordance with an aspect of the present invention, a method is presented for preparing a protein hydrolysate from a proteinaceous material in which a proteinaceous material is contacted under aqueous conditions with a proteolytic enzyme combination having an exopeptidase specific for peptides having a proline in the penultimate N-terminus. In preferred embodiments, the exopeptidase is specific for peptides having as an N-terminus a five amino acid sequence of X-Pro-Gln-Gln-Pro- wherein X is the amino terminal amino acid and can be any naturally occurring amino acid, Pro is proline and Gln is glutamine.

Preferably, the exopeptidase has a sequence having at least 70% sequence identity to one of MalPro11 (SEQ ID NO:1), MciPro4 (SEQ ID NO:2), TciPro1 (SEQ ID NO:3), FvePro4 (SEQ ID NO: 4), and SspPro2 (SEQ ID NO:5) or an active fragment thereof. More preferably, the exopeptidase has a sequence with at least 80% sequence identity to one of MalPro11 (SEQ ID NO:1), MciPro4 (SEQ ID NO:2), TciPro1 (SEQ ID NO:3), FvePro4 (SEQ ID NO: 4), and SspPro2 (SEQ ID NO:5) or an active fragment thereof. Still more preferably, the exopeptidase has a sequence with at least 85% sequence identity to one of MalPro11 (SEQ ID NO:1), MciPro4 (SEQ ID NO:2), TciPro1 (SEQ ID NO:3), FvePro4 (SEQ ID NO: 4), and SspPro2 (SEQ ID NO:5) or an active fragment thereof. In yet more preferred embodiments, the exopeptidase has a sequence with at least 90% sequence identity to one of MalPro11 (SEQ ID NO:1), MciPro4 (SEQ ID NO:2), TciPro1 (SEQ ID NO:3), FvePro4 (SEQ ID NO: 4), and SspPro2 (SEQ ID NO:5) or an active fragment thereof.

Still more preferably, the exopeptidase has a sequence with at least 95% sequence identity to one of MalPro11 (SEQ ID NO:1), MciPro4 (SEQ ID NO:2), TciPro1 (SEQ ID NO:3), FvePro4 (SEQ ID NO: 4), and SspPro2 (SEQ ID NO:5) or an active fragment thereof. In still more preferred embodiments, the exopeptidase has a sequence with at least 99% sequence identity to one of MalPro11 (SEQ ID NO:1), MciPro4 (SEQ ID NO:2), TciPro1 (SEQ ID NO:3), FvePro4 (SEQ ID NO: 4), and SspPro2 (SEQ ID NO:5) or an active fragment thereof. In the most preferred embodiments, the exopeptidase has a sequence according to one of MalPro11 (SEQ ID NO:1), MciPro4 (SEQ ID NO:2), TciPro1 (SEQ ID NO:3), FvePro4 (SEQ ID NO: 4), and SspPro2 (SEQ ID NO:5) or an active fragment thereof.

In preferred embodiments of the present invention, the proteolytic enzyme mixture has a second exopeptidase. Preferably, the second exopeptidase is an aminopeptidase. More preferably, the aminopeptidase has a sequence with at least 70% sequence identity to one of SEQ ID NO:10, SEQ ID NO:11, SEQ ID NO:12, SEQ ID NO:13, SEQ ID NO:14, SEQ ID NO:15, SEQ ID NO:16, SEQ ID NO:17 and SEQ ID NO:28 or an aminopeptidase active fragment thereof. Still more preferably, the aminopeptidase has a sequence with at least 80% sequence identity to one of SEQ ID NO:10, SEQ ID NO:11, SEQ ID NO:12, SEQ ID NO:13, SEQ ID NO:14, SEQ ID NO:15, SEQ ID NO:16, SEQ 11) NO:17 and SEQ ID NO:28 or an aminopeptidase active fragment thereof. Yet more preferably, the aminopeptidase has a sequence with at least 85% sequence identity to one of SEQ ID NO:10, SEQ ID NO:11, SEQ ID NO:12, SEQ ID NO:13, SEQ ID NO:14, SEQ ID NO:15, SEQ ID NO:16, SEQ ID NO:17 and SEQ ID NO:28 or an aminopeptidase active fragment thereof. Still more preferably, the aminopeptidase has a sequence with at least 90% sequence identity to one of SEQ ID NO:10, SEQ ID NO:1, SEQ ID NO:12, SEQ ID NO:13, SEQ ID NO:14, SEQ ID NO:15, SEQ ID NO:16. SEQ ID NO:17 and SEQ ID NO:28 or an aminopeptidase active fragment thereof

In still more preferred embodiments, the aminopeptidase has a sequence with at least 95% sequence identity to one of SEQ ID NO:10, SEQ ID NO:11, SEQ ID NO:12, SEQ ID NO:13. SEQ ID NO:14, SEQ ID NO:15, SEQ ID NO:16, SEQ ID NO:17 and SEQ ID NO:28 or an aminopeptidase active fragment thereof. Yet more preferably, the aminopeptidase has a sequence with at least 99% sequence identity to one of SEQ ID NO:10, SEQ ID NO:11, SEQ ID NO:12, SEQ ID NO:13, SEQ ID NO:14, SEQ ID NO:15, SEQ ID NO:16, SEQ ID NO:17 and SEQ ID NO:28 or an aminopeptidase active fragment thereof. Still more preferably, the aminopeptidase has a sequence according to one of SEQ ID NO:10, SEQ ID NO:11, SEQ ID NO:12. SEQ ID NO:13, SEQ ID NO:14, SEQ HD NO:15, SEQ ID NO:16, SEQ ID NO:17 and SEQ ID NO:28 or an aminopeptidase active fragment thereof. In the most preferred embodiments, the aminopeptidase has a sequence according to SEQ ID NO:10 or an aminopeptidase active fragment thereof.

In other preferred embodiments of the present invention, the proteolytic enzyme mixture also has an endopeptidase. Preferably, the endopeptidase has a sequence with at least 70% sequence identity to one of SEQ ID NO:18, SEQ ID NO:19, SEQ ID NO:20, SEQ ID NO:21, SEQ ID NO:22. SEQ ID NO:23, SEQ ID NO:24, SEQ ID NO:25 SEQ ID NO:26, and SEQ ID NO:27 or an endopeptidase active fragment thereof. More preferably, the endopeptidase has a sequence with at least 80% sequence identity to one of SEQ ID NO:18, SEQ ID NO:19, SEQ ID NO:20, SEQ ID NO:21, SEQ ID NO:22, SEQ ID NO:23, SEQ ID NO:24, SEQ ID NO:25 SEQ ID NO:26, and SEQ ID NO:27 or an endopeptidase active fragment thereof. Still more preferably, the endopeptidase has a sequence with at least 85% sequence identity to one of SEQ ID NO:18, SEQ ID NO:19, SEQ ID NO:20, SEQ ID NO:21, SEQ ID NO:22, SEQ ID NO:23, SEQ ID NO:24, SEQ ID NO:25 SEQ ID NO:26, and SEQ ID NO:27 or an endopeptidase active fragment thereof. Yet more preferably, the endopeptidase has a sequence with at least 90% sequence identity to one of SEQ ID NO:18, SEQ ID NO:10, SEQ ID NO:20, SEQ ID NO:21, SEQ ID NO:22, SEQ ID NO:23, SEQ ID NO:24, SEQ ID NO:25 SEQ ID NO:26, and SEQ ID NO:27 or an endopeptidase active fragment thereof. In still more preferred embodiments, the endopeptidase has a sequence with at least 95% sequence identity to one of SEQ ID NO:18, SEQ ID NO:19, SEQ ID NO:20, SEQ ID NO:21, SEQ ID NO:22, SEQ ID NO:23, SEQ ID NO:24, SEQ ID N025 SEQ ID NO:26, and SEQ ID NO:27 or an endopeptidase active fragment thereof. Yet more preferably, the endopeptidase has a sequence with at least 99% sequence identity to one of SEQ ID NO:18, SEQ ID NO:19, SEQ ID NO:20, SEQ ID NO:21, SEQ ID NO:22, SEQ ID NO:23, SEQ ID NO:24, SEQ ID NO:25 SEQ ID NO:26, and SEQ ID NO:27 or an endopeptidase active fragment thereof. In the most preferred embodiments, the endopeptidase has a sequence according to one of SEQ ID NO:18, SEQ ID NO:19. SEQ ID NO:20, SEQ ID NO:21, SEQ ID NO:22, SEQ ID NO:23, SEQ ID NO:24, SEQ ID NO:25 SEQ ID NO:26, and SEQ ID NO:27 or an endopeptidase active fragment thereof.

In preferred embodiments of the present invention, the proteinaceous material is a vegetable derived protein, an animal derived protein, a fish derived protein, an insect derived protein or a microbial derived protein. Preferably, the proteinaceous material comprises gluten, soy protein, milk protein, egg protein, whey, casein, meat, hemoglobin or myosin.

In other preferred embodiments, the proteolytic enzyme mixture has at least an exopeptidase specific for peptides having a proline in the penultimate N-terminus, a second exopeptidase and an endopeptidase as described above. Preferably, these enzymes are used to treat the proteinaceous material at the same time. In other preferred embodiments, these enzymes are used at different times.

In preferred embodiments of the instant invention, the method for producing a protein hydrolysate is for producing hydrolysates having elevated levels of glutamic acid. According to this aspect of the present invention, the proteolytic enzyme mixture has a glutaminase Preferably, the glutaminase has a sequence with at least 70% sequence identity to SEQ ID NO:29 or a glutaminase active fragment thereof. More preferably, the glutaminase has a sequence with at least 80% sequence identity to SEQ ID NO:29 or a glutaminase active fragment thereof. Still more preferably, the glutaminase has a sequence with at least 85% sequence identity to SEQ ID NO:29 or a glutaminase active fragment thereof. In yet more preferred embodiments, the glutaminase has a sequence with at least 90% sequence identity to SEQ ID NO:29 or a glutaminase active fragment thereof. Still more preferably, the glutaminase has a sequence with at least 95% sequence identity to SEQ ID NO:29 or a glutaminase active fragment thereof. In yet more preferred embodiments, the glutaminase has a sequence with at least 99% sequence identity to SEQ ID NO:29 or a glutaminase active fragment thereof. In the most preferred embodiments, the glutaminase has a sequence according to SEQ ID NO:29 or a glutaminase active fragment thereof.

According to this aspect of the present invention, the proteinaceous material is gluten.

In other preferred embodiments, the method for producing a protein hydrolysate is for producing hydrolysates having elevated levels of proline.

In other aspect of the present invention, a protein hydrolysate is presented produced according to any of the methods disclosed above.

In other aspect of the present invention, a food product is presented having a protein hydrolysate as described above.

### EXAMPLES

### Example 1 Cloning of Fungal X-Pro Proteases

Two fungal strains, Melanocarpus albomyces CBS177.67 (GICC #2522192) and Malbrancheae cinamonea CBS 343.55 (GICC #2518670), were selected as potential sources of enzymes which may be useful in various industrial applications. Melanocarpus albomyces CBS177.67 and Malbrancheae cinamonea CBS 343.55 were purchased from CBS-KNAW Fungal Biodiversity Centre (Uppsalalaan 8, 3584 CT Utrecht, the Netherlands). Chromosomal DNA was sequenced using the Illumina's next generation sequencing technology and two fungal X-Pro proteases were identified after annotation: MalPro11 from Melanocarpus albomyces CBS177.67 and MciPro4 from Malbrancheae cinamonea CBS 343.55. The full-length protein sequences of MalPro11 and MciPro4 are shown in SEQ ID NO: 1 and SEQ ID NO: 2, respectively.

Three fungal strains (Trichoderma citrinoviride TUCIM 6016, Fusarium verticillioides 7600 and Stagonospora sp. SRC1lsM3a) listed in JGI database (https://genome.jgi.doe.gov/portal/) were selected as potential sources of enzymes which may be useful in various industrial applications. A BLAST search (Altschul et al., J Mol Biol, 215: 403-410, 1990) led to the identification of three proteases: TciPro1 from Trichoderma citrinoviride TUCIM 6016, FvePro4 from Fusarium verticillioides 7600 and SspPro2 from Stagonospora sp. SRC1lsM3a. The full-length protein sequence of TciPro1 (JGI strain ID: Trici4, Protein ID: 1136694), FvePro4 (JGI strain ID: Fusve2, Protein ID: 4472) and SspPro2 (JGI strain ID: Stasp1, Protein ID: 303285) are set forth as SEQ ID NO: 3, SEQ ID NO: 4 and SEQ ID NO: 5, respectively.

### Example 2 Expression of Identified Fungal X-Pro Proteases

The DNA sequences encoding full length MalPro11, MciPro4 or TciPro1, following an additional 5′ DNA fragment (SEQ ID NO: 6), were chemically synthesized and inserted into a Trichoderma reesei expression vector pGXT (the same as the pTTTpyr2 vector as described in published PCT Application WO2015/017256, incorporated by reference here). The resulting plasmids were labeled as pGXT-MalPro11, pGXT-MciPro4 and pGXT-TciPro1. Each individual expression vector was then transformed into a suitable Trichoderma reesei strain (described in published PCT application WO 05/001036) using protoplast transformation (Te'o et al. (2002) J. Microbiol. Methods 51:393-99). Transformants were selected on a medium containing acetamide as a sole source of nitrogen. After 5 days of growth on acetamide plates, transformants were collected and subjected to fermentation in 250 mL shake flasks in defined media containing a mixture of glucose and sophorose.

The DNA sequences encoding truncated FvePro4 (SEQ ID NO: 7) and truncated SspPro2 (SEQ ID NO: 8) was chemically synthesized and inserted into the Bacillus subtilis expression vector p2JM103BBI (Vogtentanz, Protein Expr Purif, 55: 40-52, 2007) yielding plasmids pGXB-FvePro4 and pGXB-SspPro2, respectively. Each individual expression vector was transformed into a suitable B. subtilis strain and the transformed cells spread onto Luria Agar plates supplemented with 5 ppm chloramphenicol. Colonies were selected and subjected to fermentation in a 250 mL shake flask with a MOPS based defined medium.

To purify MalPro11, MciPro4 and TciPro1, each clarified culture supernatant was concentrated and added ammonium sulfate to a final concentration of 1 M. The solution was loaded onto a HiPrep™ Phenyl FF 16/10 column pre-equilibrated with 20 mM NaAc (pH5.0) supplemented with additional 1 M ammonium sulfate (Buffer A). The target protein was eluted from the column with 0.25 M ammonium sulfate. The corresponding fractions were pooled, concentrated and exchanged buffer into 20 mM Tris (pH8.0) (Buffer B), using a VivaFlow 200 ultra-filtration device (Sartorius Stedim). The resulting solution was applied to a HiPrep™ Q HP 16/10 column pre-equilibrated with Buffer B. The target protein was eluted from the column with 0.3 M NaCl. The fractions containing active protein were then pooled and concentrated via the 10K Amicon Ultra devices, and stored in 40% glycerol at −20° C. until usage.

To purify FvePro4 and SspPro2, each clarified culture supernatant was concentrated and added ammonium sulfate to the final concentration of 1M. The solution was loaded onto a HiPrep™ Phenyl FF 16/10 column pre-equilibrated with 20 mM NaPi (pH7.0) supplemented with additional 1 M ammonium sulfate (Buffer A). The target protein flowed through from the column. The solution was pooled, concentrated and exchanged buffer into 20 mM Tris (pH8.0) (Buffer B), using a VivaFlow 200 ultra-filtration device (Sartorius Stedim). The resulting solution was applied to a HiPrep™ HP 16/10 column pre-equilibrated with Buffer B. The target protein was eluted from the column with 0.2 M NaCl. The active fractions were pooled, added ammonium sulfate to the final concentration of 1.2 M. The solution was loaded onto a HiPrep™ Phenyl HP 16/10 column pre-equilibrated with 20 mM NaPi (pH7.0) supplemented with additional 1.2 M ammonium sulfate. The target protein was eluted from the column with a gradient elution mode from 1.2 to 0.6 M ammonium sulfate. The fractions containing active protein were then pooled and concentrated via the 10K Amicon Ultra devices, and stored in 40%/glycerol at −20° C. until usage

### Example 3 Proteolytic Activity of Purified Fungal X-Pro Proteases

The proteolytic activity of purified proteases (MalPro11, MciPro4, TciPro1, FvePro4 and SspPro2) was carried out in 50 mM Tris-HCl buffer (pH 7.5), using Phenylalanine-Proline (Phe-Pro) (GL Biochem, Shanghai) or Serine-Proline (Ser-Pro) (GL Biochem, Shanghai) as the substrate. Prior to the reaction, the enzyme was diluted with water to specific concentrations. The dipeptide substrate (Phe-Pro or Ser-Pro) was dissolved in 50 mM Tris-HCl buffer (pH 7.5, supplemented with 0.05 mM CoCl₂) to a final concentration of 10 mM. To initiate the reaction, 90 μL of 10 mM dipeptide (Phe-Pro or Ser-Pro) was added to the non-binding 96-MTP (Corning Life Sciences, #3641) and incubated at 50° C. for 5 min at 600 rpm in a Thermomixer, followed by the addition of 10 μL of the diluted enzyme sample (or water alone as the blank control). After 20 min incubation in a Thermomixer at 50° C. and 600 rpm, the protease reaction was terminated by heating at 95° C. for 10 min.

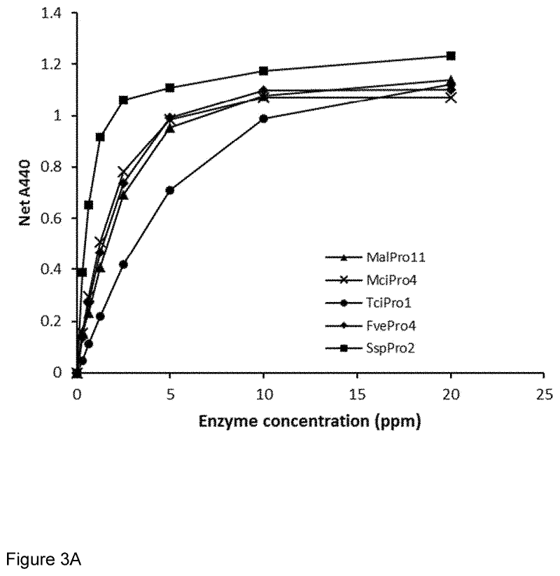

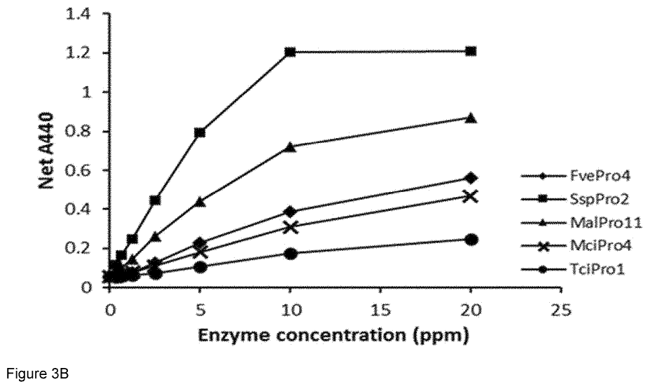

As detected by the ninhydrin reaction, the production of free Pro hydrolyzed from dipeptide (Phe-Pro or Ser-Pro) was applied to show the proteolytic activity. Prior to the reaction, ninhydrin (Sigma, #151173) was dissolved in 100% ethanol to a final concentration of 5% (w/v). To initiate the ninhydrin reaction, 40 μL of 1M sodium acetate (pH 2.8) was first mixed with 10 μL of 5% ninhydrin solution in a 96-MTP PCR plate (Axygen, PCR-96M2-HS-C), followed by the addition of 50 μL of aforementioned protease reaction solution. The whole mixture was then incubated in a Thermo cycler (BioRad) at 95° C. for 15 min. After adding 100 μL of 75% ethanol, the absorbance of the resulting solution was measured at 440 nm (A₄₄₀) using a SpectraMax 190. Net A₄₄₀ was calculated by substracting the A₄₄₀ of the blank control from that of the enzyme sample, and then plotted against different protein concentrations (from 0.3125 ppm to 20 ppm). The results are shown in FIGS. 3A and B. Each value was the mean of duplicate assays with variance less than 5%. The proteolytic activity is therefore shown as Net A₄₄₀. The proteolytic assay with Phe-Pro (FIG. 3A) or Ser-Pro (FIG. 3B) as the substrate indicates that MalPro11, MciPro4, TciPro1, FvePro4 and SspPro2 are all active proteases.

### Example 4 pH Profile of Purified Fungal X-Pro Proteases

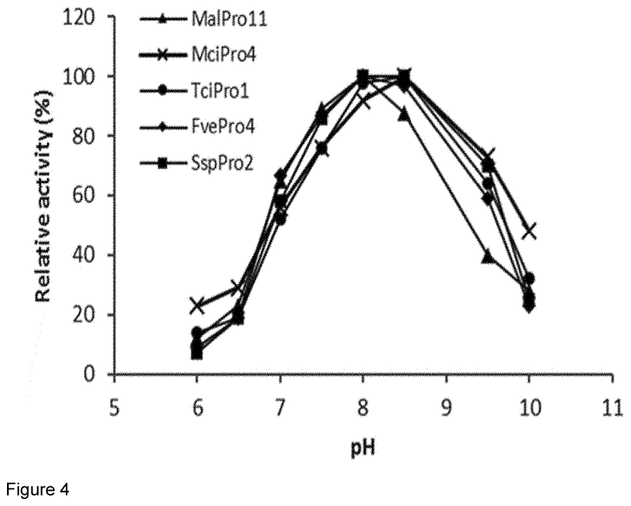

With Phe-Pro dipeptide as the substrate, the pH profile of purified proteases (MalPro11, MciPro4, TciPro1, FvePro4 and SspPro2) was studied in 25 mM Bis-tris propane buffer with different pH values (ranging from pH 6 to 10). Prior to the assay, 45 μL of 50 mM Bis-tris propane buffer with a specific pH value (supplemented with 0.1 mM CoCl₂) was first mixed with 45 μL of 20 mM Phe-Pro (dissolved in water) in a 96-MTP, and then 10 μL of water diluted enzyme (12.5 ppm for MalPro11, 25 ppm for MciPro4, 12.5 ppm for TciPro1, 12.5 ppm for FvePro4, 6.25 ppm for SspPro2, or water alone as the blank control) was added. The reaction was performed and analyzed as described in Example 3. Enzyme activity at each pH was reported as the relative activity, where the activity at the optimal pH was set to be 100%. The pH values tested were 6, 6.5, 7, 7.5, 8, 8.5, 9.5 and 10. Each value was the mean of duplicate assays with variance less than 5%. As shown in FIG. 4, the optimal pH for MalPro11, MciPro4, TciPro1, FvePro4 or SspPro2 is 8, 8.5, 8.5, 8 or 8, respectively.

### Example 5 Temperature Profile of Purified Fungal X-Pro Proteases

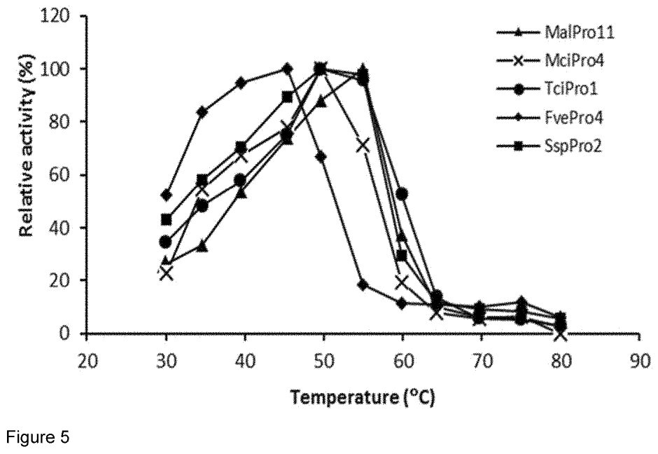

The temperature profile of purified proteases (MalPro11, MciPro4, TciPro1, FvePro4 and SspPro2) was analyzed in 50 mM Tris-HCl buffer (pH 7.5) using the Phe-Pro dipeptide as the substrate. Prior to the reaction, 90 μL of 10 mM Phe-Pro dipeptide dissolved in 50 mM Tris-HCl buffer (pH 7.5, supplemented with 0.05 mM CoCl₂) was added in a 200 μL PCR tube, which was subsequently incubated in a Thermal Cycler (BioRad) at desired temperatures (i.e. 30-80° C.) for 5 min. After the incubation, 10 μL of water diluted enzyme (12.5 ppm for MalPro11, 25 ppm for MciPro4, 12.5 ppm for TciPro1, 12.5 ppm for FvePro4, 6.25 ppm for SspPro2 or water alone as the blank control) was added to the substrate solution to initiate the reaction. Following 20 min incubation in the Thermal Cycler at different temperatures, the reaction was quenched and analyzed as described in Example 3. The activity was reported as the relative activity, where the activity at the optimal temperature was set to be 100%. The tested temperatures are 30, 35, 40, 45, 50, 55, 60, 65, 70, 75 and 80° C. Each value was the mean of duplicate assays with variance less than 5%. As shown in FIG. 5, the optimal temperature for MalPro11, MciPro4, TciPro1, FvePro4 or SspPro2 is 55, 50, 50, 45 or 50° C.; respectively.

### Example 6 Thermostability of Purified Fungal X-Pro Proteases

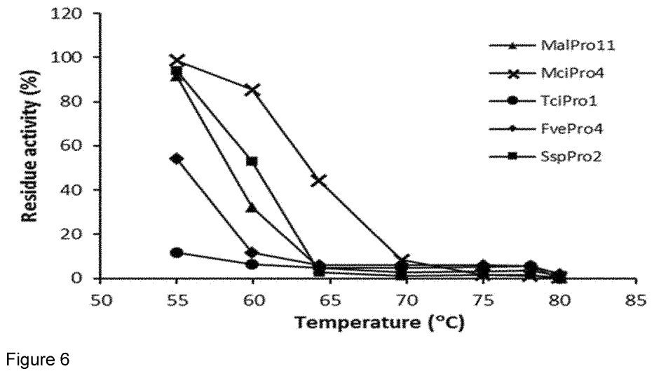

Prior to the thermostability test, the Phe-Pro dipeptide substrate was dissolved in 50 mM Tris-HCl buffer (pH 7.5, supplemented with 0.05 mM CoCl₂) to a final concentration of 10 mM. The purified proteases (MalPro11, MciPro4, TciPro1, FvePro4 and SspPro2) were diluted in 0.2 mL water to a final concentration of 200 ppm, and subsequently incubated at different temperatures (4, 55, 60, 65, 70, 75, 80° C.) for 5 min. After the incubation, each enzyme solution was further diluted with water into specific concentration (12.5 ppm for MalPro11, 25 ppm for MciPro4, 12.5 ppm for TciPro1, 12.5 ppm for FvePro4, 6.25 ppm for SspPro2 or water alone as the blank control). To measure the proteolytic activity, 10 μL of the resulting enzyme solution was mixed with 90 μL of substrate solution; and the reaction was carried out and analyzed as described in Example 3. The activity was reported as the residue activity, where the activity of enzyme sample incubated at 4° C. was set to be 100%. Each value was the mean of duplicate assays with variance less than 5%. As shown in FIG. 6, all proteases lost their activities after 5 min incubation at 70, 75 and 80° C.; and except for MciPro4, all other four also lost their activities after 5 min incubation at 65° C.

### Example 7 Pentapeptide Hydrolysis Analyses of Purified Fungal X-Pro Proteases

The proteolytic activity of purified proteases (MalPro11, MciPro4, TciPro1, FvePro4 and SspPro2) on pentapeptide Gln-Pro-Gln-Gln-Pro (GL Biochem, Shanghai) (SEQ ID NO: 9) was carried out in 50 mM Tris-HCl buffer (pH 7.5). Prior to the reaction, the enzyme was diluted with water to 200 ppm. The pentapeptide substrate was dissolved in 50 mM Tris-HCl buffer (pH 7.5, supplemented with 0.05 mM CoCl₂) to a final concentration of 10 mM. To initiate the reaction, 90 μL of 10 mM pentapeptide solution was added to the non-binding 96-MTP (Corning Life Sciences, #3641) and incubated at 50° C. for 5 min at 600 rpm in a Thermomixer, followed by the addition of 10 μL of the diluted enzyme sample (or water alone as the blank control). After 1 hr incubation in a Thermomixer at 50° C. and 600 rpm, the protease reaction was terminated by heating at 95° C. for 10 min.

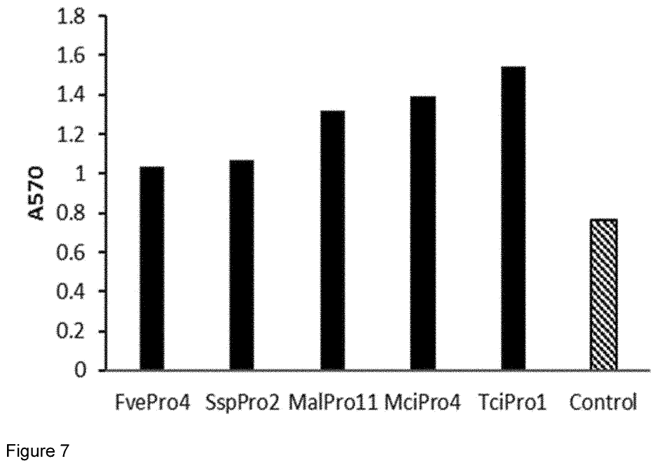

The ninhydrin reaction detecting the primary amine was applied to demonstrate the pentapeptide hydrolysis. Prior to the reaction, the ninhydrin solution was prepared containing 2% ninhydrin (w/v), 0.5 M sodium acetate, 40% ethanol and 0.2% fructose (w/v). To initiate the reaction, 90 μL of ninhydrin solution was mixed with 10 μL of aforementioned protease reaction solution in a 96-MTP PCR plate. The whole mixture was then incubated in a Thermo cycler at 95° C. for 15 min. After adding 100 μL of 75% ethanol, the absorbance of the resulting solution was measured at 570 nm (A₅₇₀) using a SpectraMax 190. The results are shown in FIG. 7. Each value was the mean of duplicate assays with variance less than 5%. The increment of A₅₇₀ for those protease samples, when compared to the blank control indicates that all purified proteases are capable of hydrolyzing pentapeptide Gln-Pro-Gln-Gln-Pro.

### Example 8: Preparation and Analysis of Gluten Pre-Hydrolysates

A substrate containing water soluble gluten peptides and amino acids was obtained by a modified version of the method described in Schlichtherle-Cerny and Amado (2002). The following was mixed in a 100 mL screw cap bottle: 6.4 g Gluten (Sigma-Aldrich, Copenhagen Denmark), 0.123 g AcPepN2, 0.6 g glutaminase SD-C100S (Amano, Nagoya Japan) 63 mg FoodPro® Alcaline protease (DuPont® Industrial Biosciences, Brabrand Denmark), 1.73 g NaCl (Analytical grade, Fischer Scientific, Roskilde Denmark) and 24.3 g water. The bottle was incubated in a thermo-block with magnetic stirring at 600 rpm and 55° C. for 18 hours. Subsequently the enzymes were inactivated by heating to 95° C. for 10 min, centrifuged for 5 min at 4600 rpm and the supernatant filtered through 0.45 μm syringe filters.

For N-terminal sequence determination of residual peptides the gluten pre-hydrolysate was filtered through a 0.2 μm syringe filter and 2 μL was loaded on a PPSQ-31B protein sequenator from Shimadzu. A mix of 25 pmol of all 20 common amino acids was made and used as standard. The retention times and areas of peaks for the amino acids in the standard were used to identify and quantify amino acids released after each step of the Edman cycler. From the results, a consensus sequence for the N-terminal of the residual peptides could be derived. This consensus sequence is: XPQQP, where X is any amino acid, P is proline and Q is glutamine. Furthermore, the results showed that 73% of the residual peptides had proline in the penultimate position.

Nano LC-MS/MS analyses were performed using a Dionex UltiMate® 3000 RSLCnano LC (Thermo Scientific) interfaced to an Orbitrap Fusion mass spectrometer (Thermo Scientific). 1 μL of each sample was loaded onto a 2 cm trap column (100 μm i.d., 375 μm o.d., C18, 5 μm reversed phase particles) connected to a 15 cm analytical column (75 μm i.d., 375 μm o.d., packed with Reprosil C18, 3 μm reversed phase particles (Dr. Maisch GmbH, Ammerbuch-Entringen)) with a pulled emitter. Separation was performed at a flow rate of 300 nL/min using a 37 minutes gradient of 5-53% Solvent B (H₂O/CH₃CN/TFE/HCOOH (100/800/100/1) v/v/v/v) into the nano-electrospray ion source (Thermo Scientific). The Orbitrap Fusion instrument was operated in a data-dependent MS/MS mode. The peptide masses were measured by the Orbitrap (MS scans were obtained with a resolution of 120.000 at m/z 200), and as many ions as possible from the most intense peptide m/z were selected and subjected to fragmentation within 1.6 seconds, using (Higher-energy collisional dissociation) HCD in the linear ion trap (LTQ). Dynamic exclusion was enabled with a list size of 500 masses, duration of 40 seconds, and an exclusion mass width of ±10 ppm relative to masses on the list.

The RAW files were processed and searched against Uniprot Green Plants using Proteome Discoverer 2.0 and a local mascot server. The areas of all identified Peptides were estimated using the build-in Area detection module in Proteome Discoverer 2.0.

An essential tool in evaluating the amount of Gln bound in residual peptides from the gluten hydrolysis was the Q-area. Q-area=Qₙ*Area, where Qₙ is the number of Gln residues in a peptide and Area is the area under the curve of the chromatographic peak that results from that specific peptide.

The results showed that one specific sequence of amino acids or “motif”, XPQQP, was in common for a large proportion of the peptides detected. Based on Q area, it was estimated that peptides carrying this sequence motif in the N-terminus was holding approximately 60% of residual glutamine.

In conclusion: Two independent analytical techniques show that the N-terminal of the residual peptides in the gluten pre-hydrolysate has the consensus sequence XPQQP.

### Example 9: Test of X-ProAP's on Gluten Pre-Hydrolysate

General procedure: The reaction mix consisted of 250 μL gluten pre-hydrolysate, 11.8 μL 50 mg/mL glutaminase, 10.2 μL μL AcPepN2 and 98 μg X-ProAP. MilliQ water was added to a total volume of 310 or 415 μL. The total volume was always constant in an experiment but varied from experiment to experiment depending on the protein concentration of the X-ProAP's used. Reference samples contained glutaminase but neither AcPepN2 nor X-ProAP. Total volume was the same as for the rest of the samples in the experiment.

All reaction mixtures were made in Eppendorf tubes. The tubes were incubated in an Eppendorf mixer at 50° C. and 800 rpm. At specified timepoints aliquots of 80 μL were taken and mixed with 20 μL 2.5M TCA (Fischer Scientific Roskilde Denmark) to stop further reaction. Glutamic acid concentration in hydrolysates was quantified using Enzymatic L-glutamic acid kit from R-BIOPHARM, Darmstadt, Germany. The method was downscaled for use in 96-well plates, otherwise carried out according to manufacturer instructions. TCA/sample mix was diluted further 400 times (total dilution factor=500) in MilliQ water prior to analysis.

Degree of hydrolysis (DH) was determined based on the o-phthaldialdehyde (OPA; Fischer Scientific, Roskilde Denmark) assay according to the method described by Nielsen et al. (Nielsen, Petersen et al. 2001). The average MW of amino acids was determined by total amino acid analysis (carried out at Eurofins, Vejen, Denmark). Based on this h, was calculated to 7.6 mmol per g of gluten protein.

Amino acid and peptide distribution was analyzed using size exclusion chromatography (SEC). The system used was from ThermoFisher Scientific, Hørsholm, Denmark and consisted of a Dionex UltiMate 3000 solvent rack, pump and autosampler with a Dionex Corona ultra RS charged aerosol detector (CAD), A Superdex™ Peptide 10/300 GL column (from Merck, Copenhagen, Denmark). Chromeleon® version 7.2 was used for instrument control and data processing. The mobile phase was composed of 20% acetonitrile (ACN) and 0.1% trifluoroacetic acid (TFA; Fischer Scientific, Roskilde Denmark) in MilliQ water. All samples were diluted 10 times in mobile phase and filtered using 0.2 μm PVDF filter plates (material #3504, CORNING Kennebunk ME, USA) prior to injection. Injection volume was 10 μL and flow rate was 0.500 m/min for 55 min.

The reference sample included in all experiments contained gluten pre-hydrolysate and glutaminase. It was exposed to the same treatment as all other samples. For ease of comparison between different runs, the reference sample is set to contain 100% glutamic acid (formed during the pre-hydrolysis step). All other results are given in % relative to the reference sample. Other samples contain the same as the reference, with addition of AcPepN2 and/or X-ProAP.

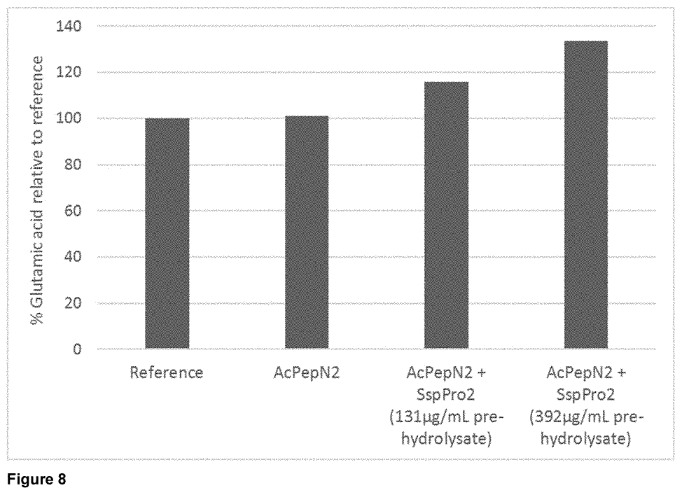

FIG. 8 shows the effect of increasing doses of SspPro2 on the glutamic acid yield. Two doses of SspPro2 were tested: 131 μg/mL and 392 μg/mL of pre-hydrolysate. This resulted in 16% and 34% increase in glutamic acid, relative to the reference, respectively. Under the given conditions, AcPepN2 alone did not give any increase in glutamic acid level.

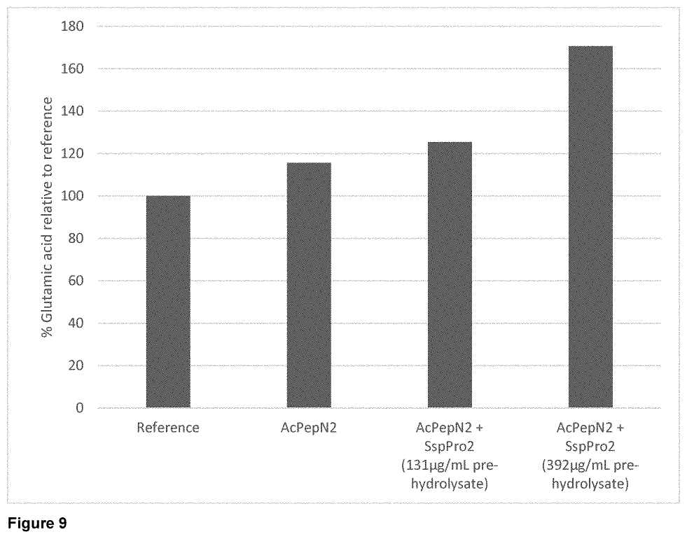

FIG. 9 shows results from the same samples as in FIG. 8 but after 26 h of incubation. In this case 131 μg/mL and 392 μg/mL of TciPro1 resulted in 25% and 71% increase in glutamic acid, relative to the reference, respectively. In this case AcPepN2 alone also gave a 16% increase in glutamic acid relative to the reference.

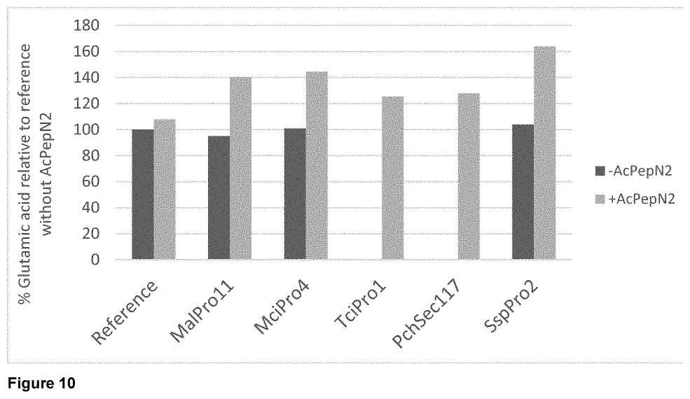

FIG. 10 shows the effect of different X-ProAP's on glutamic acid yield. The incubation time was 24 h. In this case AcPepN2 alone gave an 8% increase in glutamic acid level, relative to the reference. In combination with AcPepN2 MalPro11 MciPro4, TciPro1, PchSec117, SspPro2 gave 40%, 44%, 25%, 28% and 64% increase respectively. In contrast when MalPro11, MciPro4 and SspPro2 were tested alone (without AcPepN2) no increase in glutamic acid level was observed (not above the experimental error). The results show that AcPepN2 and the X-ProAP's tested work in synergy to release glutamic acid from the residual peptides in the pre-hydrolysate. Due to limited amount of material, TciPro1 and PchSec117 were not tested without AcPepN2.

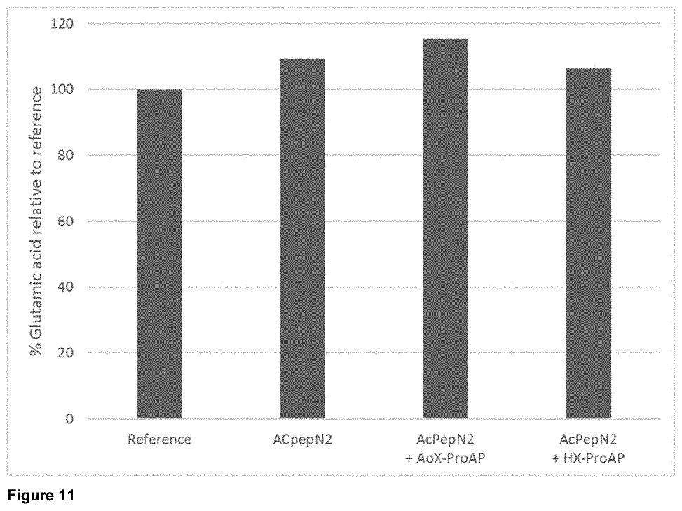

FIG. 11 shows the results from two additional X-ProAP's that were tested. They only gave negligible responses after 19 and 26 h of incubation. The results shown in FIG. 11 are after 42 hours of incubation. In this case AcPepN2 alone gave a 9% increase in glutamic acid level. AoX-ProAP and HX-ProAP gave 15% and 6% increase respectively. The difference between AcPepN2 alone and HX-ProAP is within the experimental error. Due to limited material, the dose of X-ProAP's in this case was only 15 μg/mL pre-hydrolysate.

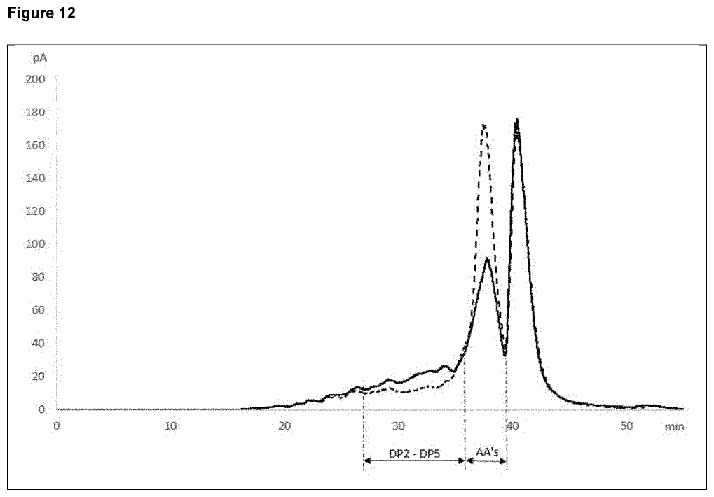

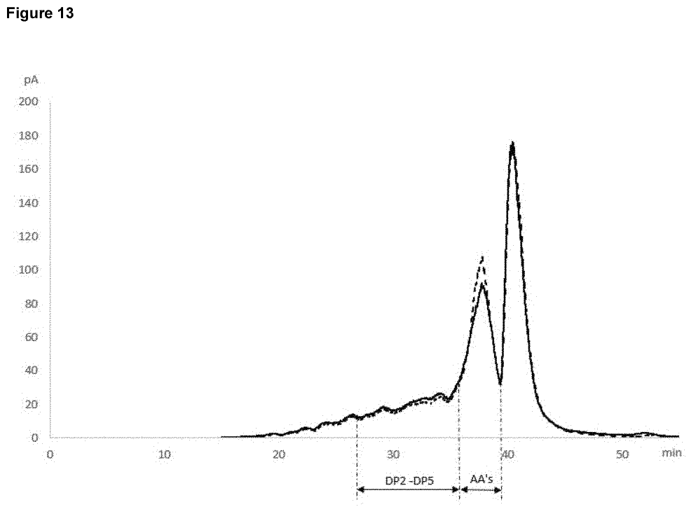

The hydrolysis profile was determined on samples from the same experiments that were used for the glutamic acid results in FIG. 8-11. Two examples are given below. In FIG. 12 the hydrolysis profile of the AcPepN2 sample (solid line) is compared to the profile of the sample containing AcPepN2+SspPro2 at 392 μg/mL pre-hydrolysate (dashed line). The peak area of the peak containing amino acids is 1.5 times higher for the hydrolysate made with AcPepN2+SspPro2 compared to the hydrolysate made which AcPepN2 alone. Concomitantly the DP2-5 area is reduced 1.3 times for the AcPepN2+SspPro2 hydrolysate compared to the AcPepN2-only hydrolysate. The reduction in DP2-5 area is not directly proportional to the increase in amino acid area, because the response factor of the CAD is not equal for amino acids and DP2-5 peptides. FIG. 13 shows a similar comparison of the hydrolysis profiles of the AcPepN2 sample and the sample containing HX-ProAP. The increase in amino acids caused by HX-ProAP is very modest. In line with the observation that this treatment did not increase Gln-levels.

### Example 10: Test of X-ProAP's on Gluten Protein Slurry

A pre-hydrolysate is not a requirement for production of glutamic acid from gluten protein. SspPro2 was tested in a setup where all components, including all enzymes, were mixed at the onset of the experiment.

A scaled down version of the method described in Schlichtherle-Cerny and Amado (2002) was used. Following was mixed in a 20 mL Wheaton vial: 2.13 g Gluten, 33 mg AcPepN2, 21 mg FoodPro® Alkaline Protease, 0.2 g glutaminase, 1 mg SspPro2, 0.58 g NaCl and approximately 8 g water. The amount of water was adjusted so that the total weight of all ingredients equalled 10.5 g. The Wheaton vials were incubated in a thermo-block with magnetic stirring at 600 rpm and 55° C. for up to 48 hours. Aliquots of 160 μL were taken at different timepoints and stopped with 40 μL 2.5M TCA. Samples were diluted further 400 times and analyzed for glutamic acid as described in Example 9 (all suppliers of chemicals and enzymes are the same as in Example 8 and 9).

After 24 h of incubation 22% more glutamic acid was formed in the sample containing SspPro2 compared to a reference sample without X-ProAP. Notice that in this case the reference sample contains active AcPepN2 as opposed to the reference sample in the gluten pre-hydrolysate experiments, where the pre-hydrolysates were made with AcPepN2+other enzymes, which were subsequently inactivated. In the gluten slurry experiments, a reference without AcPepN2 is not meaningful.

What is claimed is:

1. A method for preparing a protein hydrolysate from a proteinaceous material which method comprises contacting the proteinaceous material under aqueous conditions with a proteolytic enzyme combination comprising an exopeptidase specific for peptides having a proline in the penultimate N-terminus.

2. The method for preparing a protein hydrolysate from a proteinaceous material according to claim 1 wherein the exopeptidase is specific for peptides having as an N-terminus a five amino acid sequence of X-Pro-Gln-Gln-Pro- wherein X is the amino terminal amino acid and can be any naturally occurring amino acid, Pro is proline and Gln is glutamine.

3. The method for preparing a protein hydrolysate from a proteinaceous material according to claim 2 wherein the exopeptidase comprises a sequence having at least 70% sequence identity to one of MalPro11 (SEQ ID NO:1), MciPro4 (SEQ ID NO:2), TciPro1 (SEQ ID NO:3), FvePro4 (SEQ ID NO: 4), and SspPro2 (SEQ ID NO:5) or an active fragment thereof

4. The method for preparing a protein hydrolysate from a proteinaceous material according to claim 3 wherein the exopeptidase comprises a sequence having at least 80% sequence identity to one of MalPro11 (SEQ ID NO:1), MciPro4 (SEQ ID NO:2), TciPro1 (SEQ ID NO:3), FvePro4 (SEQ ID NO: 4), and SspPro2 (SEQ ID NO:5) or an active fragment thereof.

5. The method for preparing a protein hydrolysate from a proteinaceous material according to claim 4 wherein the exopeptidase comprises a sequence having at least 85% sequence identity to one of MalPro11 (SEQ ID NO:1), MciPro4 (SEQ ID NO:2), TciPro1 (SEQ ID NO:3), FvePro4 (SEQ ID NO: 4), and SspPro2 (SEQ ID NO:5) or an active fragment thereof.

6. The method for preparing a protein hydrolysate from a proteinaceous material according to claim 5 wherein the exopeptidase comprises a sequence having at least 90% sequence identity to one of MalPro11 (SEQ ID NO:1), MciPro4 (SEQ ID NO:2), TciPro1 (SEQ ID NO:3), FvePro4 (SEQ ID NO: 4), and SspPro2 (SEQ ID N0:5) or an active fragment thereof.

7. The method for preparing a protein hydrolysate from a proteinaceous material according to claim 6 wherein the exopeptidase comprises a sequence having at least 95% sequence identity to one of MalPro11 (SEQ ID NO:1), MciPro4 (SEQ ID NO:2), TciPro1 (SEQ ID NO:3), FvePro4 (SEQ ID NO: 4), and SspPro2 (SEQ ID NO:5) or an active fragment thereof.

8. The method for preparing a protein hydrolysate from a proteinaceous material according to claim 7 wherein the exopeptidase comprises a sequence having at least 99% sequence identity to one of MalPro11 (SEQ ID NO:1), MciPro4 (SEQ ID NO:2), TciPro1 (SEQ ID NO:3), FvePro4 (SEQ ID NO: 4), and SspPro2 (SEQ ID NO:5) or an active fragment thereof.

9. The method for preparing a protein hydrolysate from a proteinaceous material according to claim 8 wherein the exopeptidase comprises a sequence according to one of MalPro11 (SEQ ID NO:1), MciPro4 (SEQ ID NO:2), TciPro1 (SEQ ID NO:3), FvePro4 (SEQ ID NO: 4), and SspPro2 (SEQ ID NO:5) or an active fragment thereof.

10. The method for preparing a protein hydrolysate according to any preceding claim wherein the proteolytic enzyme mixture further comprises a second exopeptidase.

11. The method for preparing a protein hydrolysate according to claim 10 wherein the second exopeptidase is an aminopeptidase.

12. The method according to claim 11 wherein the aminopeptidase comprises a sequence having at least 70% sequence identity to one of SEQ ID NO:10, SEQ ID NO:11, SEQ ID NO:12, SEQ ID NO:13, SEQ ID NO:14, SEQ ID NO:15, SEQ ID NO:16, SEQ ID NO:17 and SEQ ID NO:28 or an aminopeptidase active fragment thereof.

13. The method according to claim 12 wherein the aminopeptidase comprises a sequence having at least 80% sequence identity to one of SEQ ID NO:10, SEQ ID NO:11, SEQ ID NO:12, SEQ ID NO:13, SEQ ID NO:14, SEQ ID NO:15, SEQ ID NO:16, SEQ ID NO:17 and SEQ ID NO:28 or an aminopeptidase active fragment thereof.

14. The method according to claim 13 wherein the aminopeptidase comprises a sequence having at least 85% sequence identity to one of SEQ ID NO:10, SEQ ID NO:11, SEQ ID NO:12, SEQ ID NO:13, SEQ ID NO:14, SEQ ID NO:15, SEQ ID NO:16, SEQ ID NO:17 and SEQ ID NO:28 or an aminopeptidase active fragment thereof.

15. The method according to claim 14 wherein the aminopeptidase comprises a sequence having at least 90% sequence identity to one of SEQ ID NO:10, SEQ ID NO:11, SEQ ID NO:12, SEQ ID NO:13, SEQ ID NO:14, SEQ ID NO IS, SEQ ID NO:16, SEQ ID NO:17 and SEQ ID NO:28 or an aminopeptidase active fragment thereof.

16. The method according to claim 15 wherein the aminopeptidase comprises a sequence having at least 95% sequence identity to one of SEQ ID NO:10, SEQ ID NO:11, SEQ ID NO:12, SEQ ID NO:13, SEQ ID NO:14, SEQ ID NO:15, SEQ ID NO:16, SEQ ID NO:17 and SEQ ID NO:28 or an aminopeptidase active fragment thereof.

17. The method according to claim 16 wherein the aminopeptidase comprises a sequence having at least 99% sequence identity to one of SEQ ID NO:10, SEQ ID NO:11, SEQ ID NO:12, SEQ ID NO:13, SEQ 11 NO:14, SEQ ID NO:15, SEQ ID NO:16, SEQ ID NO:17 and SEQ ID NO:28 or an aminopeptidase active fragment thereof.

18. The method according to claim 17 wherein the aminopeptidase comprises a sequence according to one of SEQ ID NO:10, SEQ ID NO:11, SEQ ID NO:12, SEQ ID NO:13, SEQ ID NO:14, SEQ ID NO:15, SEQ ID NO:16, SEQ ID NO:17 and SEQ ID NO:28 or an aminopeptidase active fragment thereof.

19. The method according to claim 18 wherein the aminopeptidase comprises a sequence according to SEQ ID NO:10 or an aminopeptidase active fragment thereof.

20. The method for preparing a protein hydrolysate according any of the preceding claims wherein the proteolytic enzyme mixture further comprises an endopeptidase.

21. The method according to claim 20 wherein the endopeptidase comprises a sequence having at least 70% sequence identity to one of SEQ ID NO:18, SEQ ID NO:19, SEQ ID NO:20, SEQ ID NO:21, SEQ ID NO:22, SEQ ID NO:23, SEQ ID NO:24, SEQ ID NO:25 SEQ ID NO:26, and SEQ ID NO:27 or an endopeptidase active fragment thereof.

22. The method according to claim 21 wherein the endopeptidase comprises a sequence having at least 80% sequence identity to one of SEQ ID NO:18, SEQ ID NO:19, SEQ ID NO:20, SEQ ID NO:21, SEQ ID NO:22, SEQ ID NO:23, SEQ ID NO:24, SEQ ID NO:25 SEQ ID NO:26, and SEQ ID NO:27 or an endopeptidase active fragment thereof.

23. The method according to claim 22 wherein the endopeptidase comprises a sequence having at least 85% sequence identity to one of SEQ ID NO:18, SEQ ID NO:19, SEQ ID NO:20, SEQ ID NO:21, SEQ ID NO:22, SEQ ID NO:23, SEQ ID NO:24, SEQ ID NO:25 SEQ ID NO:26, and SEQ ID NO:27 or an endopeptidase active fragment thereof.

24. The method according to claim 22 wherein the endopeptidase comprises a sequence having at least 90% sequence identity to one of SEQ ID NO:18, SEQ ID NO:19, SEQ ID NO:20, SEQ ID NO:21, SEQ ID NO:22, SEQ ID NO:23, SEQ ID NO:24, SEQ ID NO:25 SEQ ID NO:26, and SEQ ID NO:27 or an endopeptidase active fragment thereof.

25. The method according to claim 23 wherein the endopeptidase comprises a sequence having at least 95% sequence identity to one of SEQ ID NO:18, SEQ ID NO:19, SEQ ID NO:20, SEQ ID NO:21, SEQ ID NO:22, SEQ ID NO:23, SEQ ID NO:24, SEQ ID NO:25 SEQ ID NO:26, and SEQ ID NO:27 or an endopeptidase active fragment thereof.

26. The method according to claim 24 wherein the endopeptidase comprises a sequence having at least 99% sequence identity to one of SEQ ID NO:18, SEQ ID NO:19, SEQ ID NO:20, SEQ ID NO:21, SEQ ID NO:22, SEQ ID NO:23, SEQ ID NO:24, SEQ ID NO:25 SEQ ID NO:26, and SEQ ID NO:27 or an endopeptidase active fragment thereof.

27. The method according to claim 25 wherein the endopeptidase comprises a sequence according to one of SEQ ID NO:18, SEQ ID NO:19, SEQ ID NO:20, SEQ ID NO:21, SEQ ID NO:22, SEQ ID NO:23, SEQ ID NO:24, SEQ ID NO:25 SEQ ID NO:26, and SEQ ID NO:27 or an endopeptidase active fragment thereof.

28. The method for preparing a protein hydrolysate according to any of the preceding claims wherein the proteinaceous material comprises a vegetable derived protein, an animal derived protein, a fish derived protein, an insect derived protein or a microbial derived protein.

29. The method for preparing a protein hydrolysate according to claim 27 wherein the proteinaceous material comprises gluten, soy protein, milk protein, egg protein, whey, casein, meat, hemoglobin or myosin.

30. The method for preparing a protein hydrolysate according to any of the preceding claims wherein the proteolytic enzyme mixture comprises at least an exopeptidase specific for peptides having a proline in the penultimate N-terminus, a second exopeptidase and an endopeptidase.

31. The method for preparing a protein hydrolysate according to claim 29 wherein the exopeptidase specific for peptides having a proline in the penultimate N-terminus corresponds to that specified by any of claims 2-9, the second exopeptidase corresponds to that specified by any of claims 11-19 and the endopeptidase corresponds to that specified by any of claims 21-26.

32. The method for preparing a protein hydrolysate according to claim 29 wherein the proteinaceous material is treated with the exopeptidase specific for peptides having a proline in the penultimate N-terminus, the second exopeptidase and the endopeptidase at the same time.

33. The method for preparing a protein hydrolysate according to claim 29 wherein the proteinaceous material is treated with the exopeptidase specific for peptides having a proline in the penultimate N-terminus, the second exopeptidase and the endopeptidase at different times.

34. The method for preparing a protein hydrolysate according to any of the preceding claims wherein the method is for producing a protein hydrolysate having elevated levels of glutamic acid.

35. The method for preparing a protein hydrolysate according to claim 33 wherein the proteolytic enzyme mixture further comprises a glutaminase.

36. The method for preparing a protein hydrolysate according to claim 34 wherein the glutaminase comprises a sequence having at least 70% sequence identity to SEQ ID NO:29 or a glutaminase active fragment thereof.

37. The method for preparing a protein hydrolysate according to claim 35 wherein the glutaminase comprises a sequence having at least 80% sequence identity to SEQ ID NO:29 or a glutaminase active fragment thereof.

38. The method for preparing a protein hydrolysate according to claim 36 wherein the glutaminase comprises a sequence having at least 85% sequence identity to SEQ ID NO:29 or a glutaminase active fragment thereof.

39. The method for preparing a protein hydrolysate according to claim 37 wherein the glutaminase comprises a sequence having at least 90% sequence identity to SEQ ID NO:29 or a glutaminase active fragment thereof.

40. The method for preparing a protein hydrolysate according to claim 34 wherein the glutaminase comprises a sequence having at least 95% sequence identity to SEQ ID NO:29 or a glutaminase active fragment thereof.

41. The method for preparing a protein hydrolysate according to claim 34 wherein the glutaminase comprises a sequence having at least 99% sequence identity to SEQ ID NO:29.

42. The method for preparing a protein hydrolysate according to claim 34 wherein the glutaminase comprises a sequence according to SEQ ID NO:29 or a glutaminase active fragment thereof.

43. The method for preparing a protein hydrolysate according to any of claims 33-41 wherein the proteinaceous material comprises gluten.

44. The method for preparing a protein hydrolysate according to any of claims 1-32 wherein the method is for producing a protein hydrolysate having elevated levels of proline.

45. A protein hydrolysate produced according to a method according to any of the preceding claims.

46. A food product comprising a protein hydrolysate according to claim 44.
